(19) United States
(12) Patent Application Publication
(10) Pub. No.: US 2013/0148782 A1
(43) Pub. Date: Jun. 13, 2013

(54) RADIATION IMAGING APPARATUS AND OPERATION METHOD THEREOF

(71) Applicant: Fujifilm Corporation, Tokyo (JP)

(72) Inventor: Takashi TAJIMA, Ashigarakami-gun (JP)

(73) Assignee: FUJIFILM CORPORATION, Tokyo (JP)

(21) Appl. No.: 13/688,487

(22) Filed: Nov. 29, 2012

(30) Foreign Application Priority Data
Dec. 8, 2011 (JP) 2011-269122
Sep. 4, 2012 (JP) 2012-194580

(51) Int. Cl. A61B 6/00

(52) U.S. Cl. CPC A61B 6/542; A61B 6/585
USPC 378/62

(57) ABSTRACT

An X-ray imaging system performs pre-exposure and main exposure to take a single X-ray image. In the pre-exposure, an AEC circuit reads out a dose detection signal outputted from a detection pixel, and compares an integral value of the dose detection signal with a threshold value. When the integral value has reached the threshold value, the AEC circuit stops X-ray emission. A main exposure condition determination unit determines main irradiation time, being one item of a main exposure condition, based on irradiation time in the pre-exposure, an integral dose in the pre-exposure, and a necessary dose required for production of the X-ray image. After that, the main exposure is performed immediately with the determined main irradiation time. Normal pixels perform a charge accumulation operation continuously from the start of the pre-exposure to the end of the main exposure, and the X-ray image is produced from accumulated electric charge.

### BACKGROUND OF THE INVENTION

1. Field of the Invention

The present invention relates to a radiation imaging apparatus and an operation method of the radiation imaging apparatus.

2. Description Related to the Prior Art

In a medical field, a radiation imaging system, for example, an X-ray imaging system using X-rays is widely known. The X-ray imaging system is constituted of an X-ray generating apparatus for generating the X-rays, and an X-ray imaging apparatus that takes an X-ray image by reception of the X-rays passed through a patient's body. The X-ray generating apparatus includes an X-ray source for emitting the X-rays to the patient's body, a source controller for controlling the operation of the X-ray source, and an emission switch for issuing an emission start signal of the X-rays to the source controller. The X-ray imaging apparatus includes an X-ray image detecting device that detects the X-ray image by conversion of the X-rays passed through the patient's body into an electric signal, and a console that controls the operation of the X-ray image detecting device and saves and displays the X-ray image.

The X-ray imaging system that uses a flat panel detector (FPD) as the X-ray image detecting device becomes widespread. The FPD has an imaging area having a matrix of pixels each of which accumulates signal charge in accordance with an X-ray dose incident thereon. The FPD accumulates the signal charge on a pixel-by-pixel basis. The accumulated signal charge is read out from each pixel to a signal processing circuit through a switching element such as a TFT. The signal processing circuit converts the signal charge into a voltage signal. Thereby, the X-ray image is electrically detected.

An electronic cassette that has the FPD contained in a portable flat box-shaped housing is in practical use. The electronic cassette is mounted not only on a specific imaging stand, but also on an existing imaging stand shareable with a film cassette and an IP cassette (CR cassette). Furthermore, the electronic cassette is sometimes used with being put on a bed under the patient's body or held by the patient himself/herself, to take a radiograph of a body part that is hard to take with the stationary X-ray image detecting device. Also, the electronic cassette is sometimes brought out from a hospital for use in bedside radiography of a home-care patient or in an outside accident or natural disaster site in an emergency.

Some X-ray imaging systems have the function of automatic exposure control (AEC) to obtain a radiographic image having appropriate image quality with reduced radiation exposure to the patient. In the AEC, a dose detection sensor measures an integral dose being an integral value of an X-ray dose during X-ray emission, and the X-ray emission is stopped at the time when the integral dose has reached a target dose. The X-ray dose applied by the X-ray source is determined by a tube current-time product (mAs value), which is the product of X-ray irradiation time (in units of seconds “s”) and tube current (in units of milliamperes “mA”) that defines the amount of the X-rays to be applied from the X-ray source per unit of time. An exposure condition including the X-ray irradiation time and the tube current-time product has a rough recommendation value depending on a body portion to be imaged (chest, head, and the like), the sex and age of the patient, and the like. However, X-ray transmittance varies in accordance with the individual difference e.g. physique of the patient, so the AEC facilitates obtaining more appropriate image quality according to each individual patient.

As a method for the AEC, as described in Japanese Patent Laid-Open Publication No. 2008-086358, for example, pre-exposure and main exposure are performed to take a single X-ray image, and a main exposure condition including the X-ray irradiation time and the tube current-time product is determined based on a result of the pre-exposure. A pre-exposure condition used in the pre-exposure is determined based on the body portion to be imaged and patient information such as the sex and age of the patient.

The X-ray imaging device of the Japanese Patent Laid-Open Publication No. 2008-086358 has the dose detection sensors, which detect the X-ray dose in the pre-exposure, independent of the FPD that detects the X-ray image in the main exposure. In the pre-exposure, only the dose detection sensors are actuated, and the X-ray dose is detected to determine the main exposure condition. In the main exposure, the FPD is actuated to detect the X-ray image.

In the Japanese Patent Laid-Open Publication No. 2008-086358, the FPD is not actuated in the pre-exposure. The X-ray dose applied during the pre-exposure is used only for the determination of the main exposure condition. The pre-exposure is useful for determining the appropriate main exposure condition, but causes useless radiation exposure to the patient because the X-ray dose applied during the pre-exposure is not reflected in the X-ray image. Although the X-ray dose applied in the pre-exposure is much less than that applied in the main exposure, the radiation exposure to the patient should be as little as possible.

### SUMMARY OF THE INVENTION

An object of the present invention is to provide a radiation imaging apparatus and an operation method thereof in which a radiation dose applied to a patient during pre-exposure is reflected without waste in a radiographic image for use in diagnosis.

To achieve the above and other objects of the present invention, a radiation imaging apparatus according to the present invention includes an FPD and a control unit. The FPD has an imaging area having an arrangement of pixels. Each pixel accumulates electric charge in accordance with a radiation dose received by each pixel from a radiation generating apparatus through an object. The control unit controls the operation of the FPD such that the FPD performs a charge accumulation operation continuously from the start of pre-exposure to the end of main exposure and performs a readout operation after the end of the main exposure. In the charge accumulation operation, the pixels accumulate the electric charge. In the readout operation, the accumulated electric charge is read out from the pixels and the radiographic image for use in the diagnosis is outputted.

The radiation imaging apparatus further includes a dose detection sensor, an AEC circuit, a timer, a main exposure condition determination unit, and a communication unit. The dose detection sensor detects the radiation dose received by the imaging area. The AEC circuit judges based on output of the dose detection sensor whether or not an integral dose being an integral value of the radiation dose has reached a target dose. When the integral dose is judged to have reached the target dose, the AEC circuit issues an emission stop signal to stop radiation emission from the radiation generating apparatus. The timer measures pre-irradiation time from the start of the radiation emission to the issue of the emission stop signal in the pre-exposure. The main exposure condition determination unit determines the main exposure condition based on the pre-irradiation time and a predetermined necessary dose required for production of the radiographic image. The communication unit transmits the emission stop signal and the main exposure condition to the radiation generating apparatus.

The main exposure condition determination unit preferably determines, as the main exposure condition, main irradiation time being radiation irradiation time in the main exposure or a main tube current-time product being a product of tube current in the main exposure and the main irradiation time. To determine the main irradiation time or the main tube current-time product, the main exposure condition determination unit preferably divides a pre-integral dose being the integral value of the radiation dose in the pre-exposure by the pre-irradiation time or a tube current-time product in the pre-exposure to obtain an integral dose per unit of time or per unit of the tube current-time product, and then divides a subtraction result of the pre-integral dose from the necessary dose by the integral dose per unit of time or per unit of the tube current-time product.

The radiation imaging apparatus may further include an amplifier and a gain setting unit. The amplifier amplifies both a voltage signal read out from the pixels in the readout operation and the output of the dose detection sensor in the pre-exposure with different gains. The gain setting unit changes gain setting of the amplifier. The gain setting unit sets the gain to be used for amplifying the output of the dose detection sensor higher than the gain to be used for amplifying the voltage signal.

The gain setting unit preferably has the function of calculating at least one of the pre-integral dose and a main integral dose being the integral value of the radiation dose during the main exposure. The gain setting unit preferably determines the gain used in the readout operation based on the pre-integral dose, the main integral dose, or a comparison result between the necessary dose and the sum of the pre-integral dose and the main integral dose.

The main exposure condition determination unit may determine tube voltage used in the main exposure based on a comparison result between the pre-irradiation time and a predetermined irradiation time threshold value. In a case where the main exposure condition determination unit changes the tube voltage used in the main exposure from the tube voltage used in the pre-exposure, the dose detection sensor and the AEC circuit may perform exposure control in the main exposure.

The radiation imaging apparatus may further include a memory for storing which of the plurality of dose detection sensors distributed in the imaging area to use on a basis of a body portion to be imaged, an input device for designating the body portion, and a detection sensor selector for selecting one or more of the dose detection sensors in accordance with the body portion designated by the input device.

The detection sensor selector may select, out of the plurality of dose detection sensors, the dose detection sensor present in at least one of an area to be most noticed in diagnosis and a directly exposed area to which radiation is directly applied.

The pixels may include a normal pixel and a detection pixel. The normal pixel produces and accumulates signal charge in accordance with the radiation dose and outputs the signal charge to a signal line through a switching element. The detection pixel functions as the dose detection sensor. The detection pixel is directly connected to the signal line without through the switching element or is provided with another switching element driven independently of the switching element of the normal pixel.

In the pre-exposure, the detection pixel preferably performs a dose detection operation for detecting the radiation dose. In the main exposure, the detection pixel preferably performs the charge accumulation operation together with the normal pixel, and accumulated the signal charge is preferably read out from both of the normal pixel and the detection pixel after the completion of the charge accumulation operation.

The radiation imaging apparatus may further include a correction circuit for correcting an output value of the detection pixel in accordance with a ratio Ta/Tb between a charge accumulation period Ta of the normal pixel starting from the pre-exposure and a charge accumulation period Tb of the detection pixel starting from the main exposure. The control unit produces the radiographic image based on the corrected output value of the detection pixel and an output value of the normal pixel.

The radiation imaging apparatus may further include a preview image generator for producing a preview image based on output of the dose detection sensor in the pre-exposure, and a console for receiving the preview image from the preview image generator and displaying the preview image. The preview image generator preferably transmits the preview image to the console before the FPD performs the readout operation. The preview image generator preferably transmits the preview image to the console, while the FPD is performing the charge accumulation operation in the main exposure.

The radiation imaging apparatus may further include a movement detection circuit. The preview image generator may produce a pre-preview image based on the output of the dose detection sensor in the pre-exposure, and produce a main preview image based on the output of the dose detection sensor in the main exposure. The movement detection circuit may compare the pre-preview image with the main preview image, to detect presence or absence of body movement of the object between the pre-exposure and the main exposure. When the movement detection circuit detects the presence of the body movement, the movement detection circuit may perform at least one of a step of outputting the emission stop signal to stop the radiation emission from the radiation generating apparatus and a step of warning the presence of the body movement.

After the communication unit transmits the main exposure condition, the main exposure is preferably started immediately.

The communication unit may adopt a wireless method. The radiation imaging apparatus may be an electronic cassette having the FPD contained in a portable housing.

An operation method of the radiation imaging apparatus includes the steps of accumulating electric charge in the pixels continuously from the start of the pre-exposure to the end of the main exposure; reading out the accumulated electric charge from the pixels after the end of the main exposure: and producing the radiographic image for use in diagnosis from the accumulated electric charge.

The operation method may further include the steps of emitting radiation from the radiation generating apparatus to the object in the pre-exposure; detecting, by the dose detection sensor, the radiation dose received by the imaging area through the object; judging whether or not the integral dose being an integral value of the radiation dose has reached the target dose based on output of the dose detection sensor; when the integral dose is judged to have reached the target dose, issuing the emission stop signal to stop the emission of the radiation from the radiation generating apparatus; measuring the pre-irradiation time from the start of the pre-exposure to the issue of the emission stop signal; determining the main exposure condition based on the pre-irradiation time and the predetermined necessary dose required for production of the radiographic image; and transmitting the main exposure condition to the radiation generating apparatus.

According to the present invention, the FPD performs the charge accumulation operation continuously from the start of the pre-exposure to the end of the main exposure. After the completion of the main exposure, the FPD performs the readout operation in which the accumulated electric charge is read out from the pixels and the radiographic image for use in diagnosis is outputted. Therefore, the radiation dose that is applied to the object during pre-exposure is reflected without waste in the radiographic image for use in diagnosis.

### BRIEF DESCRIPTION OF THE DRAWINGS

For more complete understanding of the present invention, and the advantage thereof, reference is now made to the subsequent descriptions taken in conjunction with the accompanying drawings, in which:

FIG. 1 is a schematic view of an X-ray imaging system;

FIG. 2 is a block diagram showing the structure of a source controller;

FIG. 3 is a graph showing the relation between a received dose and time;

FIG. 4 is a perspective view of an electronic cassette;

FIG. 5 is a block diagram of an FPD;

FIG. 6A is an explanatory view showing an example of the distribution of detection pixels;

FIG. 6B is an explanatory view showing another example of the distribution of the detection pixels;

FIG. 7 is a block diagram of an AEC circuit;

FIG. 8 is a table of exposure conditions set up in a console;

FIG. 9 is a block diagram of the console;

FIG. 10 is a block diagram showing the functions of the console and an information flow;

FIG. 11 is a timing chart of an X-ray imaging process;

FIG. 12 is a flowchart of the X-ray imaging process;

FIG. 13 is a block diagram of a signal processing circuit having a gain setting unit;

FIG. 14 is a flowchart of the X-ray imaging process in the case of changing tube voltage used in main exposure;

FIG. 15 is a block diagram of an FPD having detection pixels each of which is provided with a TFT driven independently of that of a normal pixel;

FIG. 16 is a flowchart of the X-ray imaging process in which the detection pixels start performing charge accumulation operation at the moment when the AEC circuit judges that an integral dose has reached a target dose;

FIG. 17 is an explanatory view of a method for correcting an output voltage signal from the detection pixels;

FIG. 18 is a block diagram of an FPD having a preview image generator;

FIG. 19 is an explanatory view showing an example of a preview image;

FIG. 20 is a block diagram of an FPD having a body movement detection circuit; and

FIG. 21 is an explanatory view of a warning window displayed on a monitor of the console in a case where the body movement detection circuit detects body movement of the patient.

### DESCRIPTION OF THE PREFERRED EMBODIMENTS

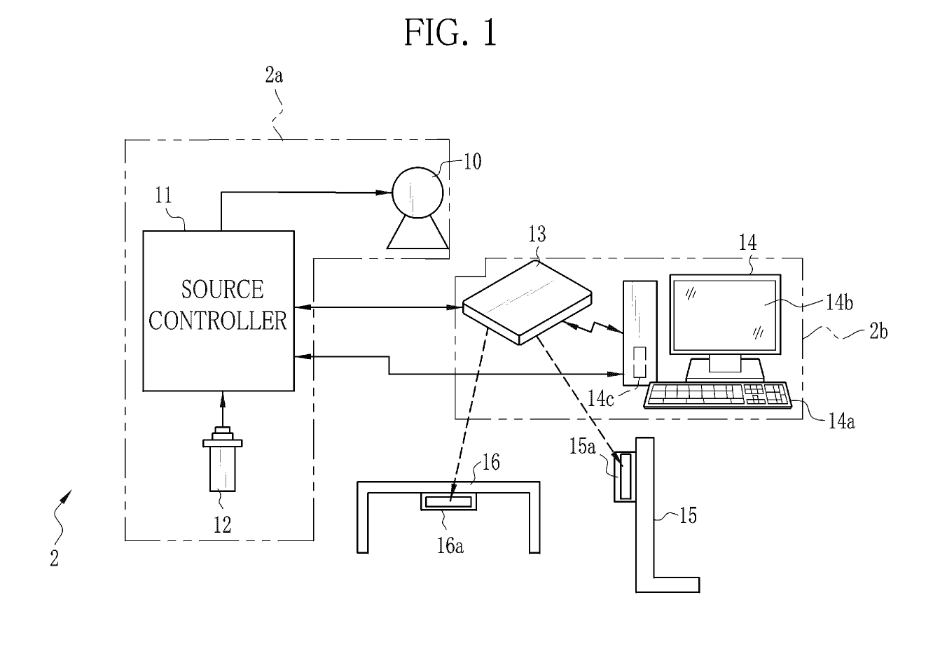

As shown in FIG. 1, an X-ray imaging system 2 is constituted of an X-ray source 10, a source controller 11, an emission switch 12, an electronic cassette 13, a console 14, and an imaging stand 15, and an imaging table 16. The X-ray source 10 contains an X-ray tube for emitting X-rays. The source controller 11 controls the operation of the X-ray source 10. The emission switch 12 commands the start of warming-up and the start of X-ray emission to the X-ray source 10. The electronic cassette 13 detects the X-rays that have passed through a patient's body (object), and outputs an X-ray image. The console 14 performs the operation control of the electronic cassette 13 and the display process of the X-ray image. The imaging stand 15 and the imaging table 16 are used in X-ray imaging of the patient who is in a standing position and a lying position, respectively. The X-ray source 10, the source controller 11, and the emission switch 12 compose an X-ray generating apparatus 2a. The electronic cassette 13 and the console 14 compose an X-ray imaging apparatus 2b. In addition to above, the X-ray imaging system 2 has a source shift mechanism (not shown) for setting the X-ray source 10 in a desired orientation and position, and the X-ray source 10 is sharable between the imaging stand 15 and the imaging table 16.

The X-ray source 10 has the X-ray tube and a collimator for limiting an irradiation field of the X-rays radiating from the X-ray tube. The X-ray tube has a cathode being a filament for emitting thermoelectrons, and an anode (target) for radiating the X-rays by collision of the thermoelectrons emitted from the cathode. The collimator is composed of, for example, four X-ray shielding lead plates disposed on each side of a rectangle so as to form a rectangular irradiation opening in its middle through which the X-rays propagate. Changing the positions of the lead plates can vary the size of the irradiation opening to limit the irradiation field.

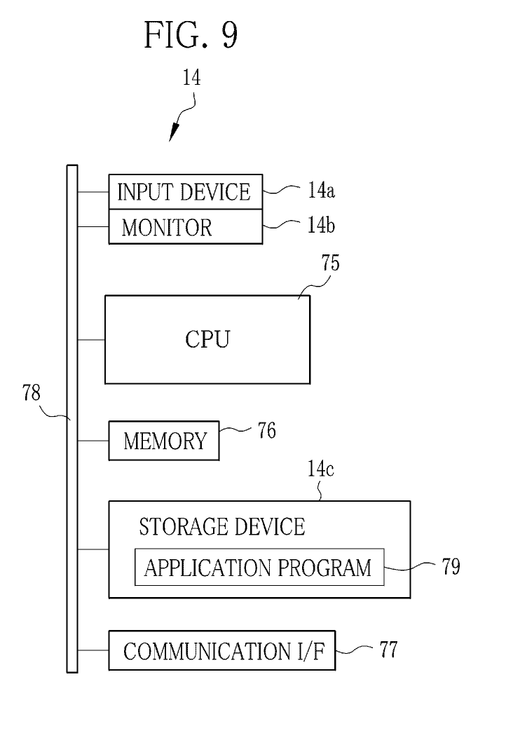

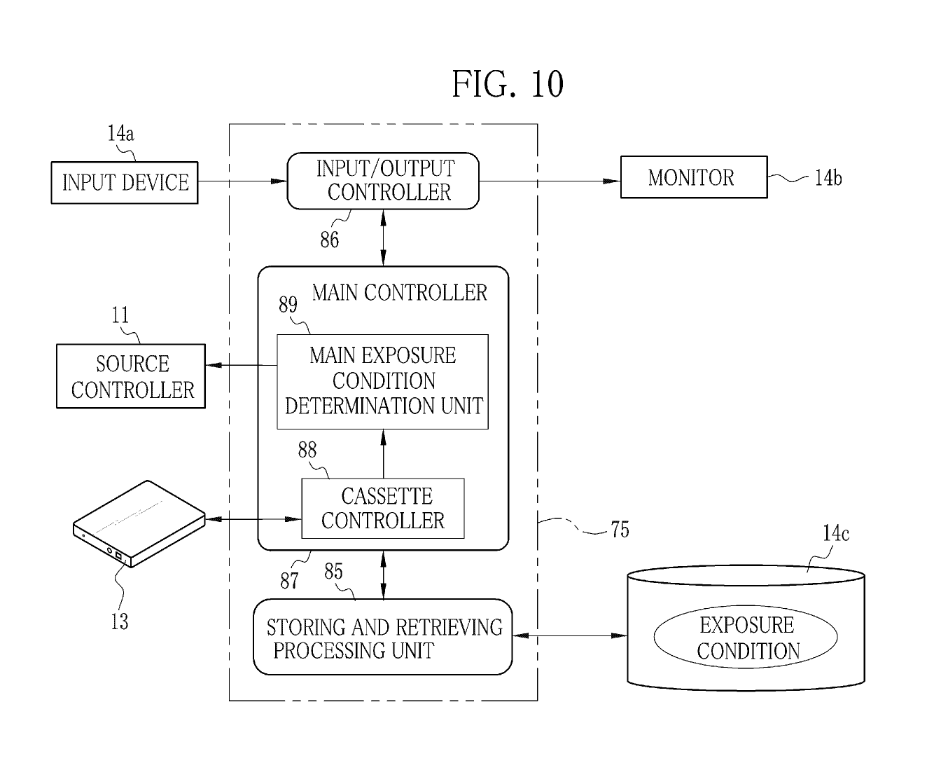

The console 14 is communicatably connected to the electronic cassette 13 in a wired or wireless method, to control the operation of the electronic cassette 13 in response to input operation by a radiological technician through an input device 14a such as a keyboard. The X-ray image from the electronic cassette 13 is displayed on a monitor 14b of the console 14, and its data is written to a storage device 14c and a memory 76 (see FIG. 9) of the console 14, or data storage such as an image storage server connected to the console 14 through a network.

The console 14 receives input of an examination order including information about the sex and age of the patient, a body portion to be imaged, an examination purpose, and the like, and displays the examination order on the monitor 14b. The examination order is inputted from an external system e.g. HIS (hospital information system) or RIS (radiography information system) that manages patient data and examination data related to radiography, or inputted manually by the radiological technician. The examination order includes an item of the body portion (object) to be imaged e.g. “head”, “chest”, “abdomen”, “hand”, “finger”, and the like. This item also includes an imaging direction e.g. “anterior”, “medial”, “diagonal”, “PA” (X-rays are applied to the object from a posterior direction), “AP” (X-rays are applied to the object from an anterior direction), and the like. The radiological technician checks the contents of the examination order on the monitor 14b, and inputs the exposure conditions corresponding to the contents of the examination order through an operation screen displayed on the monitor 14b.

The X-ray imaging system 2 performs both pre-exposure and main exposure to obtain a single X-ray image for use in diagnosis. In the pre-exposure, an X-ray dose less than that of the main exposure is applied to the object for a shorter time period than that of the main exposure, in order to determine the exposure condition (main exposure condition) used in the main exposure, and more specifically to determine irradiation time (main irradiation time), being one item of the main exposure condition, required for obtaining a desired X-ray image. In the main exposure, the X-rays are applied for the main irradiation time determined in the pre-exposure.

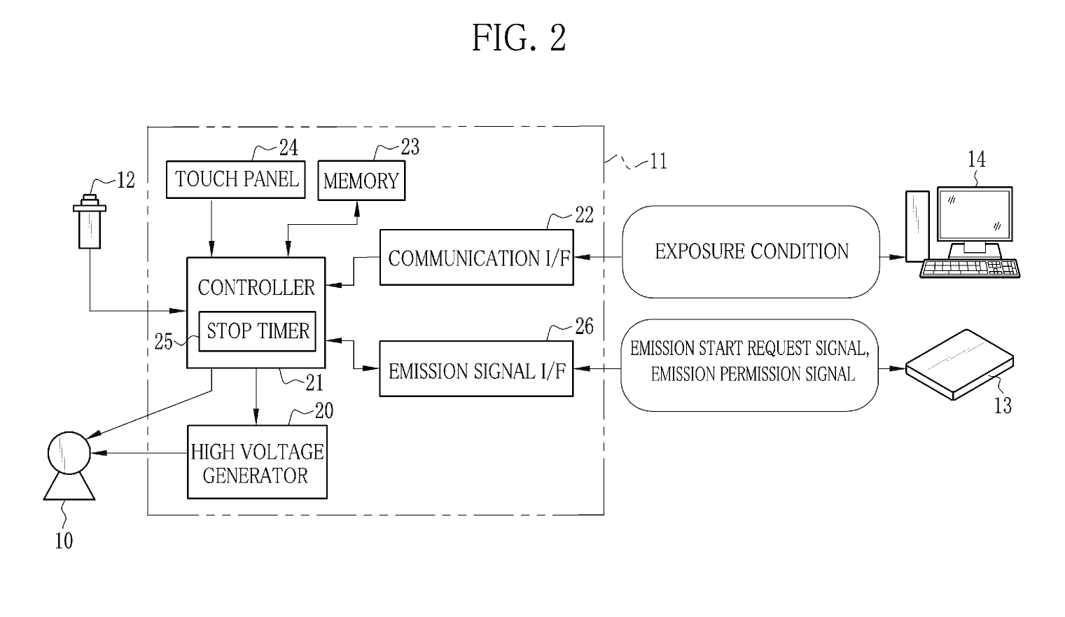

As shown in FIG. 2, the source controller 11 includes a high voltage generator 20, a controller 21, and a communication I/F 22. The high voltage generator 20 produces a high tube voltage by multiplying an input voltage using a transformer, and supplies the high tube voltage to the X-ray source 10 through a high voltage cable. The controller 21 controls the tube voltage for determining an energy spectrum of the X-rays from the X-ray source 10, a tube current for determining an X-ray irradiation amount per unit of time, and the X-ray irradiation time. The communication I/F 22 mediates transmission and reception of essential information and signals to and from the console 14.

To the controller 21, the emission switch 12, a memory 23, and a touch panel 24 are connected. The emission switch 12 is a two-step press switch operated at the time of starting the pre-exposure by the radiological technician. Upon a half press of the emission switch 12, a warm-up start signal is issued to start warming up the X-ray source 10. Upon a full press of the emission switch 12, an emission start signal is issued to start emitting the X-rays from the X-ray source 10. These signals are inputted to the controller 21 through a signal cable.

The main exposure is automatically started immediately after the determination of the main irradiation time. If the full press of the emission switch 12 is released during the main exposure, the X-ray emission is urgently stopped.

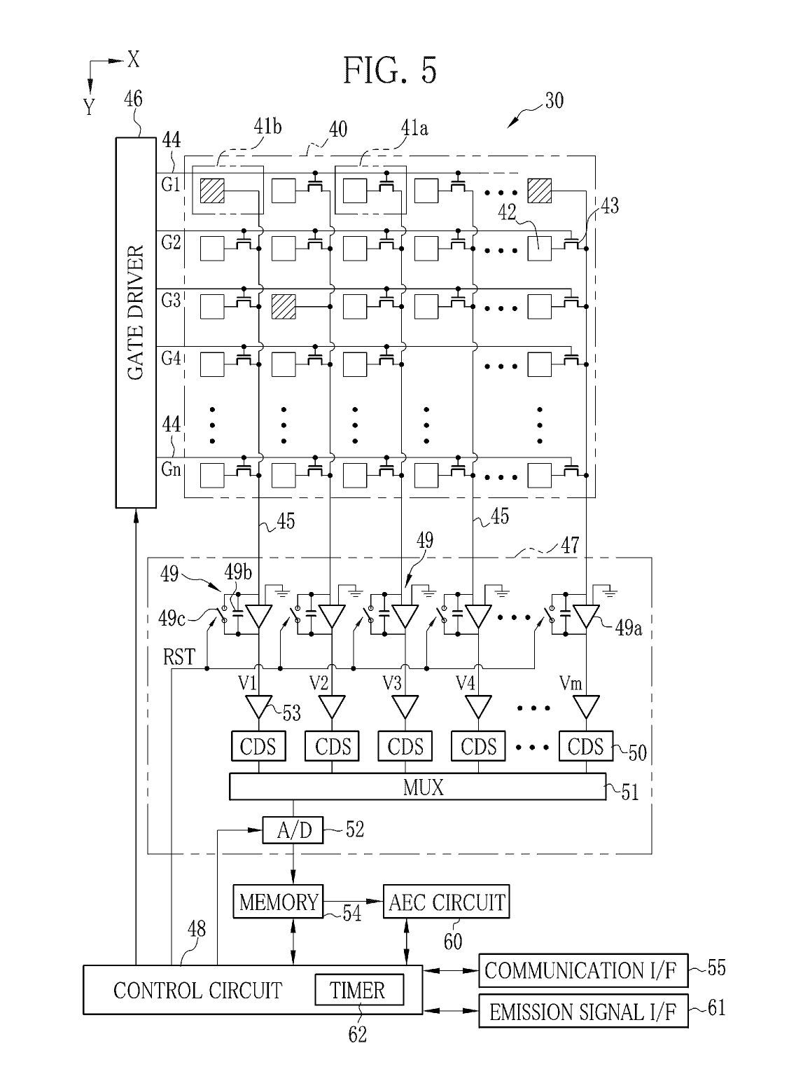

The memory 23 stores in advance several types of exposure conditions each including the tube voltage, the tube current, the irradiation time, and the like. The radiological technician manually chooses one of the exposure conditions through the touch panel 24. The tube voltage and the tube current take the same values in both the pre-exposure and the main exposure. The irradiation time set in the pre-exposure condition takes its maximum value in order to prevent a situation in which the X-ray emission is stopped before an AEC circuit 60 (see FIG. 5) of the electronic cassette 13 commands the stop of the X-ray emission and an integral dose applied during the pre-exposure becomes insufficient. On the other hand, the main irradiation time takes a value that is determined based on the pre-exposure. The controller 21 has a stop timer 25 for stopping the X-ray emission at the moment when measured time comes to the set irradiation time.

In the pre-exposure, the source controller 11 controls the X-ray emission based on the set exposure condition (pre-exposure condition) including the tube voltage, the tube current, and the irradiation time (maximum value). When the AEC circuit 60 judges that an integral dose being an integral value of a received X-ray dose has reached a target dose, the AEC circuit 60 stops the X-ray emission even if elapsed irradiation time has not yet reached the irradiation time set in the pre-exposure condition. The maximum value of the irradiation time set in the pre-exposure condition preferably differs from one body portion to another.

An emission signal I/F 26 is connected to the electronic cassette 13 with or without a cable in the case of regulating stop timing of the X-ray emission by the AEC circuit 60 based on output from detection pixels 41b (see FIG. 5). In this case, in response to the warm-up start signal from the emission switch 12 issued upon the half press of the emission switch 12, the controller 21 starts warming up the X-ray source 10. Then, the controller 21 transmits an emission start request signal through the emission signal I/F 26 to the electronic cassette 13 to ask if the X-ray emission can be started.

In response to the emission start request signal, the electronic cassette 13 checks whether or not the electronic cassette 13 itself is ready for imaging. If the electronic cassette 13 stands ready, the electronic cassette 13 issues an emission permission signal. In a case where the controller 21 has received the emission start signal from the emission switch 12 and the emission permission signal at the emission signal I/F 26, the controller 21 makes the high voltage generator 20 start supplying electric power to the X-ray source 10 to start the X-ray emission under the pre-exposure condition. Furthermore, the controller 21 stops the electric power supply from the high voltage generator 20 to the X-ray source 10 to stop the X-ray emission, as soon as the emission signal I/F 26 receives an emission stop signal issued from the electronic cassette 13 in the pre-exposure, or as soon as elapsed irradiation time measured by the stop timer 25 has reached the main irradiation time in the main exposure.

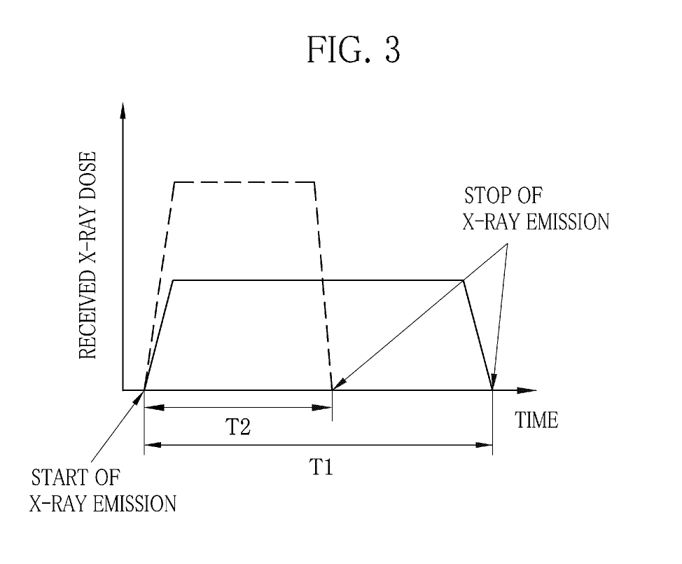

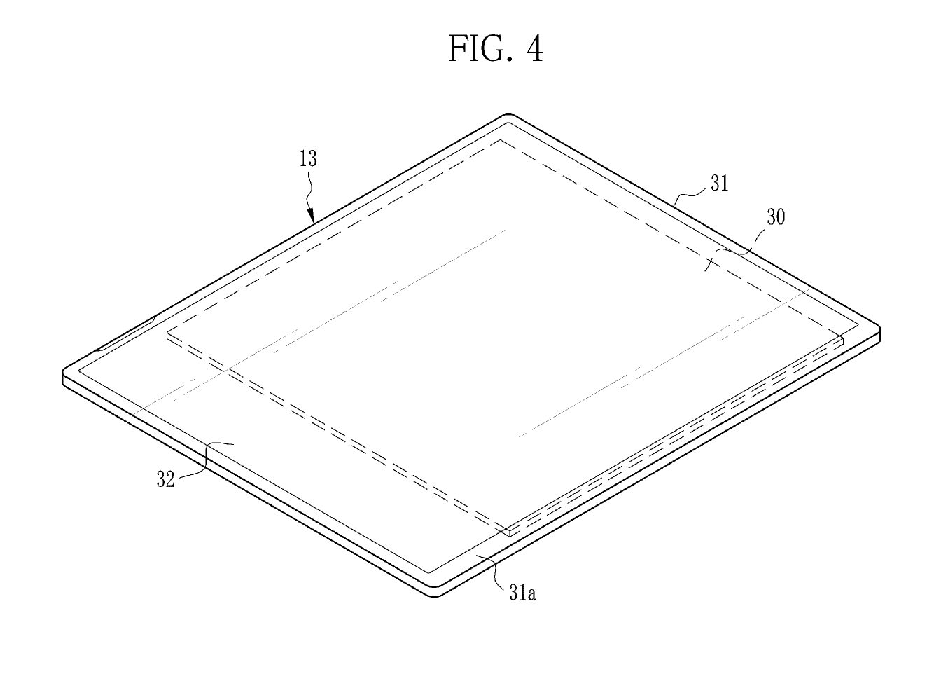

The target dose set in the pre-exposure is much less than a main X-ray dose to be applied in the main exposure. Thus, in the pre-exposure, though the irradiation time set in the source controller 11 takes its maximum value, the X-ray emission discontinues before the elapsed irradiation time comes to the maximum value, in actual fact. Before a lapse of the maximum irradiation time, the AEC circuit 60 stops the X-ray emission. In the case of emitting the X-rays with the same tube voltage and the same tube current, for example, if the thickness of the patient's body is relatively large, an X-ray dose that is received per unit of time by an imaging area 40 (see FIG. 5) of an FPD 3 (see FIGS. 4 and 5) through the object becomes small. Therefore, as shown in FIG. 3 by solid lines, irradiation time T1 to achieve a necessary integral dose becomes long. On the other hand, if the thickness of the patient's body is relatively small, as shown by broken lines, irradiation time T2 becomes short. Similarly, if the density of internal body tissue is relatively high, the irradiation time becomes long due to low X-ray transmittance. If the density of internal body tissue is relatively low, the irradiation time becomes short. In any case, the AEC circuit 60 controls the irradiation time so as to make the integral dose (the size of a trapezoidal area) equal to the target dose.

In FIG. 4, the electronic cassette 13 is constituted of the FPD 30 and a portable flat box-shaped housing 31 containing the FPD 30. The housing is made of conductive resin, for example. The housing 31 has a rectangular opening formed in its front surface 31a, and a transparent board 32 is fitted into the opening as a top plate. The transparent board 32 is made of a carbon material that has lightweight, high rigidity, and high X-ray transmittance. The housing 31 functions as an electromagnetic shield, which prevents the entrance of electromagnetic noise into the electronic cassette 13 and the radiation of the electromagnetic noise from the electronic cassette 13. In addition to the FPD 30, the housing 31 contains a battery (secondary battery) for supplying electric power with a predetermined voltage to each part of the electronic cassette 13, and an antenna for wirelessly transmitting data e.g. X-ray image data to the console 14.

The housing 31 is compatible with ISO 4090:2001 in size and shape, as with a film cassette and an IP cassette. Thus, the electronic cassette 13 can be detachably mounted on a holder 15a (see FIG. 1) of the imaging stand 15 or a holder 16a (see FIG. 1) of the imaging table 16 so as to be held in such a position that the front surface 31a of the housing 31 faces to the X-ray source 10. The source shift mechanism shifts the X-ray source 10 in accordance with which of the imaging stand 15 and the imaging table 16 is to be used. In addition, the electronic cassette 13 can be used separately from the imaging stand 15 or the imaging table 16 in a state of being put on a bed under the patient's body or held by the patient himself/herself. The electronic cassette 13 is almost the same size as the film cassette and the IP cassette, and hence can be mounted on an existing imaging stand or table shareable with the film cassette and the IP cassette.

As shown in FIG. 5, the FPD 30 has a TFT active matrix substrate and an imaging area 40 formed in this substrate. In the imaging area 40, a plurality of pixels 41 each of which accumulates electric charge in accordance with the received X-ray dose are arranged at predetermined intervals into a matrix with “n” rows (X direction) and “m” columns (Y direction). Note that, “n” and “m” are integers of 2 or more.

The FPD 30 is of an indirect conversion type, which has a scintillator (phosphor, not shown) for converting the X-rays into visible light. The pixels 41 perform photoelectric conversion of the visible light produced by the scintillator. The scintillator is made of CsI:Tl (thallium activated cesium iodide), GOS (Gd₂O₂S:Tb, terbium activated gadolinium oxysulfide), or the like. The scintillator is opposed to the entire imaging area 40 having the pixels 41. The scintillator and the TFT active matrix substrate may adopt either a PSS (penetration side sampling) method or an ISS (irradiation side sampling) method. The scintillator and the TFT active matrix substrate are disposed in this order from an X-ray incident side in the PSS method, while being disposed in reverse order in the ISS method. Note that, a direct conversion type FPD, which has a conversion layer (amorphous selenium or the like) for directly converting the X-rays into the electric charge, may be used instead.

As is widely known, the pixel 41 is composed of a photoelectric conversion element 42 that produces the electric charge (electron and hole pairs) by entry of the visible light, a capacitor (now shown) for accumulating the electric charge produced by the photoelectric conversion element 42, and a TFT 43 being a switching element.

The photoelectric conversion element 42 is composed of a semiconducting layer (of PIN type, for example) for producing the electric charge, and upper and lower electrodes disposed on the top and bottom of the semiconducting layer. The lower electrode of the photoelectric conversion element 42 is connected to the TFT 43. The upper electrode of the photoelectric conversion element 42 is connected to a bias line. The number of the bias lines coincides with the number of the rows (“n” rows) of the pixels 41. All the “n” bias lines are connected to a bias power source through a bus. The bias power source applies bias voltage to the upper electrodes of the photoelectric conversion elements 42 through the bus and the bias lines. Since the application of the bias voltage produces an electric field in the semiconducting layer, the electric charge (electron and hole pairs) produced in the semiconducting layer by the photoelectric conversion is attracted to the upper and lower electrodes, one of which has positive polarity and the other has negative polarity. Thereby, the electric charge is accumulated in the capacitor.

A gate electrode of the TFT 43 is connected to a scan line 44. A source electrode of the TFT 43 is connected to a signal line 45. A drain electrode of the TFT 43 is connected to the photoelectric conversion element 42. The scan lines 44 and the signal lines 45 are routed into a lattice. The number of the scan lines 44 coincides with the number “n” of the rows of the pixels 41, and the pixels 41 of the same row are connected to the common scan line 44. The number of the signal lines 45 coincides with the number “m” of the columns of the pixels 41, and the pixels 41 of the same column are connected to the common signal line 45. All the scan lines 44 are connected to a gate driver 46, and all the signal lines 45 are connected to a signal processing circuit 47.

The gate driver 46 drives the TFTs 43 under the control of a control circuit 48, so the FPD 30 performs a charge accumulation operation in which the pixels 41 accumulate the signal charge in accordance with the received X-ray dose, a readout operation in which the accumulated signal charge is read out from the pixels 41, and a reset operation. In the charge accumulation operation, while every TFT 43 is turned off, every pixel 41 accumulates the signal charge. In the readout operation, the gate driver 46 issues sequentially at predetermined intervals gate pulses G1 to Gn each of which drives the TFTs 43 of the same row at a time. Thereby, the scan lines 44 are activated one by one, and the TFTs 43 connected to the activated scan line 44 are turned on row by row. Upon turning on the TFT 43, the signal charge accumulated in the capacitor of the pixel 41 is read out to the signal line 45, and inputted to the signal processing circuit 47.

Dark charge occurs in the semiconducting layer of the photoelectric conversion element 42 irrespective of the presence or absence of entry of the X-rays. Due to the application of the bias voltage, the dark charge is accumulated in the capacitor of the pixel 41. The dark charge occurring in the pixels 41 becomes noise of the image data, and therefore the reset operation is carried out before the X-ray emission repeatedly at predetermined intervals to remove the dark charge. The reset operation is an operation in which the dark charge accumulated in the pixels 41 is discharged through the signal lines 45.

The reset operation adopts a sequential reset method, for example, in which the pixels 41 are reset on a row-by-row basis. In the sequential reset method, as in the case of the readout operation of the signal charge, the gate driver 46 sequentially issues the gate pulses G1 to Gn at predetermined intervals to the scan lines 44, to turn on the TFTs 43 on a row-by-row basis.

Instead of the sequential reset method, a parallel reset method or an all pixels reset method may be used. In the parallel reset method, a plurality of rows of pixels are grouped together, and sequential reset is carried out in each group so as to concurrently discharge the dark charge from the rows of the number of the groups. In the all pixels reset method, the gate pulse is inputted to every row to concurrently discharge the dark charge from every pixel. Using the parallel reset method and the all pixels reset method can reduce time required for the reset operation.

The signal processing circuit 47 is provided with integrating amplifiers 49, CDS circuits (CDSs) 50, a multiplexer (MUX) 51, an A/D converter (A/D) 52, and the like. One integrating amplifier 49 is connected to each signal line 45. The integrating amplifier 49 includes an operational amplifier 49a and a capacitor 49b connected between input and output terminals of the operational amplifier 49a. The signal line 45 is connected to one of two input terminals of the operational amplifier 49a. The other input terminal of the operational amplifier 49a is connected to a ground (GND). To the capacitor 49b, a reset switch 49c is connected in parallel. Each integrating amplifier 49 converts the electric charge inputted from the signal line 45 into each of analog voltage signals V1 to Vm by integration. An output terminal of every operational amplifier 49a is connected to the MUX 51 through another amplifier 53 and the CDS 50. An output of the MUX 51 is connected to the A/D 52.

The CDS 50, having sample hold circuits, applies correlated double sampling to an output voltage signal from the integrating amplifier 49 to remove noise, and holds the output voltage signal from the integrating amplifier 49 for a predetermined period in its sample hold circuit. The MUX 51 sequentially selects one of the CDSs 50, which are connected in parallel to one another, by an electronic switch based on an operation control signal from a shift resister (not shown), so the voltage signals V1 to Vm outputted from the CDSs 50 are inputted to the A/D 52 in series. Further another amplifier may be connected between the MUX 51 and the A/D 52.

The A/D 52 converts the inputted analog voltage signals V1 to Vm of one row into digital values, and outputs the digital values to a memory 54 contained in the electronic cassette 13. The memory 54 stores the digital values of one row with being associated with coordinates of individual pixels 41 as image data of one row of the X-ray image. Thereby, the readout operation of one row is completed.

After the MUX 51 reads out the voltage signals V1 to Vm of one row from the integrating amplifiers 49, the control circuit 48 outputs a reset pulse RST to the integrating amplifiers 49, so every reset switch 49c is turned on. Thereby, the signal charge of one row accumulated in the capacitors 49b is discharged and reset. After the reset of the integrating amplifiers 49, the reset switches 49c are turned off again. After a lapse of predetermined time from the turn off of the reset switches 49c, one of sample hold circuits of the CDSs 50 is held to sample a kTC noise component of the integrating amplifier 49. After that, the gate pulse of the next row is outputted from the gate driver 46 to start reading out the signal charge from the pixels 41 of the next row. After a lapse of predetermined time from the output of the gate pulse, the signal charge from the pixels 41 of the next row is held by another sample hold circuit of each CDS 50. By repetition of the above operation, the signal charge is read out from the pixels 41 of every row.

After the completion of the readout from every row, the image data representing the X-ray image of a single frame is stored in the memory 54. This image data is read out from the memory 54, and subjected to various types of image processing in the control circuit 48. Then, the image data is outputted to the console 14 through communication I/F 55. Thereby, the X-ray image of the object is detected.

In the reset operation, while the TFT 43 is turned on, the dark charge from the pixel 41 flows into the capacitor 49b of the integrating amplifier 49 through the signal line 45. In contrast to the readout operation, the MUX 51 does not read out the electric charge accumulated in the capacitor 49b. In synchronization with the issue of each of the gate pulses C1 to Gn, the control circuit 48 outputs the reset pulse RST. Thereby, the reset switch 49c is turned on, and the electric charge accumulated in the capacitor 49b is discharged to reset the integrating amplifier 49.

The control circuit 48 has circuits (not shown) for applying the various types of image processing such as offset correction, sensitivity correction, and defect correction to the X-ray image data stored in the memory 54. The offset correction circuit subtracts an offset correction image, which is obtained by the FPD 30 without irradiation with the X-rays, from the X-ray image on a pixel-by-pixel basis in order to remove fixed pattern noise caused by the individual difference of the signal processing circuit 47 and imaging environment. The sensitivity correction circuit, which is also called gain correction circuit, corrects variations in the sensitivity of the photoelectric conversion elements 42 of the pixels 41, variations in the output property of the signal processing circuit 47, and the like. The defect correction circuit performs linear interpolation of a pixel value of a defect pixel using a pixel value of a normal pixel around the defect pixel, based on information of the defect pixel produced in shipping or periodic inspection. The defect correction circuit also interpolates a pixel value of the detection pixel 41b used for the AEC. The console 14 may have the various image processing circuits as described above, and perform the various types of image processing.

The pixels 41 include normal pixels 41a and the detection pixels 41b. The normal pixel 41a is used in producing the X-ray image, as conventional. The detection pixel 41b, on the other hand, functions as a dose detection sensor that detects the X-ray dose received by the imaging area 40, and is used in the AEC. The detection pixels 41b are hatched in FIG. 5 as distinguished from the normal pixels 41a.

The basic structure of the photoelectric conversion element 42 and the like is exactly the same between the normal pixel 41a and the detection pixel 41b. Thus, the normal pixel 41a and the detection pixel 41b are formed by almost the same manufacturing process. The difference between the normal pixel 41a and the detection pixel 41b is that the detection pixel 41b is directly connected to the signal line 45 without through the TFT 43. Therefore, the electric charge produced in the detection pixel 41b is immediately read out to the signal line 45. The detection pixel 41b continues outputting the electric charge, even if the normal pixels 41a arranged in the same row as the detection pixel 41b have the TFTs 43 being turned off and are in the middle of the charge accumulation operation. Thus, irrespective of the state of the TFT 43, the electric charge produced in the photoelectric conversion element 42 of the detection pixel 41b always flows into the capacitor 49b of the integrating amplifier 49 through the signal line 45. During the charge accumulation operation of the normal pixels 41a, the electric charge that is produced by the detection pixel 41b and accumulated in the capacitor 49 is outputted to the A/D 52, and is converted to a digital voltage signal (hereinafter called dose detection signal) by the A/D 52.

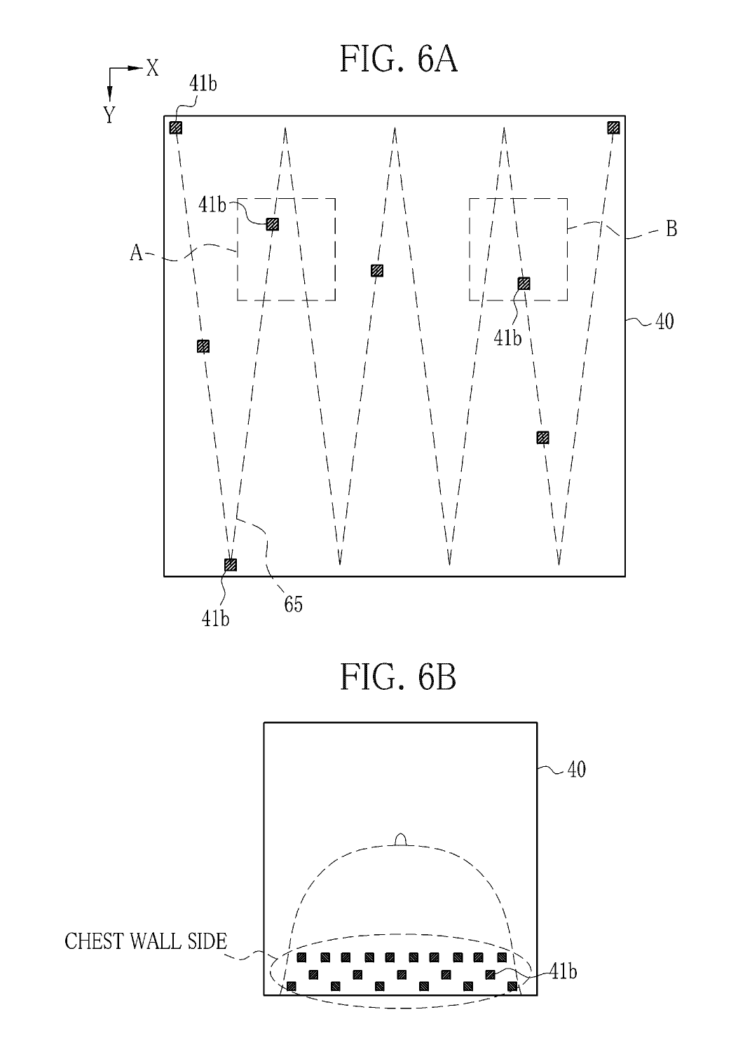

As shown in FIG. 6A, the detection pixels 41b are disposed along a zigzag line symmetric with respect to the center of the imaging area 40 as shown by broken lines, so as to be almost uniformly distributed in the imaging area 40. It is preferable that the number of the detection pixels 41b is of the order of approximately 0.01% of the number of all pixels 41. For example, one detection pixel 41b is laid out every two to three signal lines 45, and two or more detection pixels 41b are not laid out in the single signal line 45. The positions of the detection pixels 41b are already known in manufacturing the FPD 30, and the FPD 30 has a nonvolatile memory (not shown) that stores the position (coordinates) of every detection pixel 41b in advance. Note that, the disposition of the detection pixels 41b is appropriately changeable. Conversely to this embodiment, the detection pixels 41b may be disposed in a concentrated manner. For example, in a mammography device for taking radiography of a breast, as shown in FIG. 6B, the detection pixels 41b are preferably concentrated on a chest wall side.

The pre-exposure and the main exposure are carried out in series. For the purpose of reflecting the X-ray dose applied during the pre-exposure in the X-ray image read out after the completion of the main exposure, the FPD 30 starts the charge accumulation operation of the normal pixels 41a in synchronization with the start of the pre-exposure, and continues the charge accumulation operation until the completion of the main exposure. In the pre-exposure, on the other hand, the FPD 30 performs the dose detection operation for the AEC with the use of the detection pixels 41b. In other words, the FPD 30 simultaneously performs both the charge accumulation operation and the dose detection operation during the pre-exposure.

In the dose detection operation carried out in the pre-exposure, the electric charge produced in the photoelectric conversion element 42 of the detection pixel 41b flows into the capacitor 49b of the integrating amplifier 49 through the signal line 45, irrespective of the state of the TFT 43. The electric charge accumulated in the integrating amplifier 49 is outputted to the A/D 52, and is converted into the dose detection signal by the A/D 52. The dose detection signal is outputted to the memory 54.

There is one detection pixel 41b provided in a few signal lines 45. As shown in FIG. 6A, the Y-directional positions of the detection pixels 41b differ from one signal line 45 to another. The control circuit 48 stores in advance the coordinate information of each detection pixel 41b, that is, a column number of the signal line 45 having the detection pixel 41b and a row number representing the Y-directional position of the detection pixel 41b. The memory 54 stores the dose detection signals of the detection pixels 41b with being associated with the coordinate information of individual detection pixels 41b. The control circuit 48 repeats this dose detection operation for several times at a predetermined sampling rate.

The AEC circuit 60 is controlled by the control circuit 48. The AEC circuit 60 reads out from the memory 54 the dose detection signals, which are repeatedly obtained for several times at the predetermined sampling rate in the pre-exposure, and performs the AEC based on the read dose detection signals.

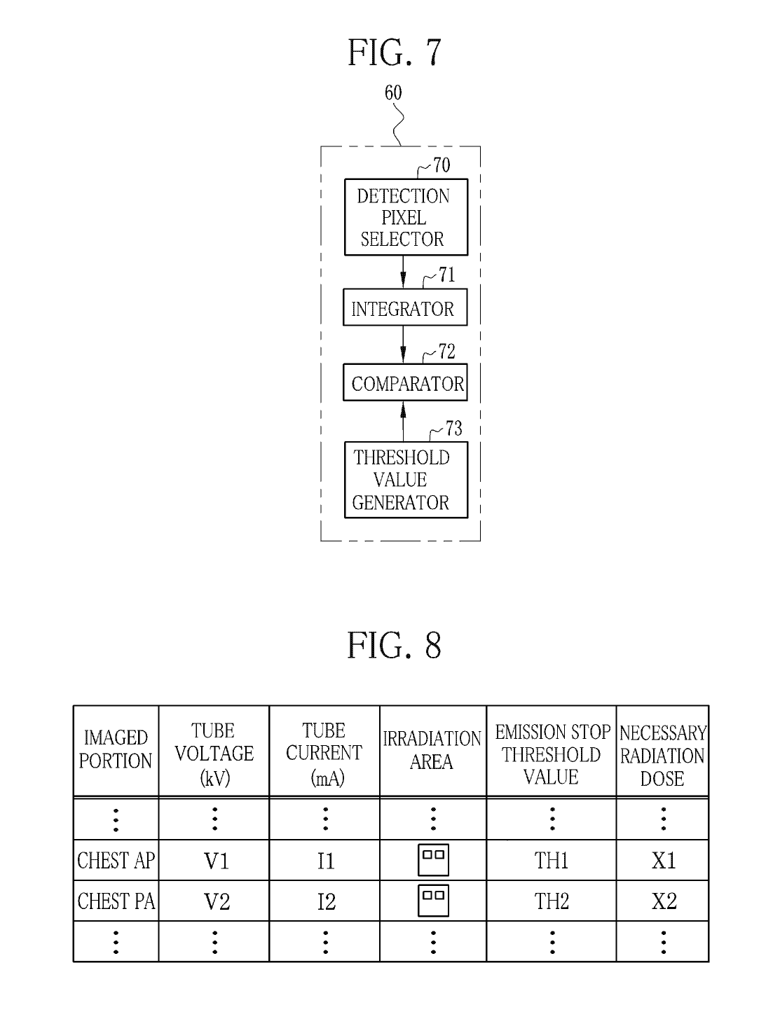

The AEC circuit 60 successively adds the dose detection signals, which are read out from the memory 54 over the plurality of times of dose detection operations, from coordinate to coordinate, to measure the integral dose received by the imaging area 40. To be more specific, as shown in FIG. 7, the AEC circuit 60 includes a detection pixel selector 70, an integrator 71, a comparator 72, and a threshold value generator 73. The detection pixel selector 70 selects which detection pixel 41b to use, out of the plurality of detection pixels 41b distributed in the imaging area 40, based on information of an irradiation area from the console 14. The integrator 71 calculates an integral value of the dose detection signal of each of one or more detection pixels 41b selected by the detection pixel selector 70. Then, the integrator 71 calculates a mean value (a mean value of the integral values of the X-ray dose received by the irradiation area) by dividing the sum of the integral values by the number of the selected detection pixels 41b. The comparator 72 compares the mean value with an emission stop threshold value (target dose) provided by the threshold value generator 73 at appropriate times. When it is judged that the mean value of the integral values of the X-ray dose exceeds the emission stop threshold value and the integral dose has reached the target dose, the comparator 72 issues the emission stop signal.

There are several ways of determining the irradiation area. For example, the imaging area 40 is equally divided into portions of predetermined size in advance, and the integral dose is obtained on a portion-by-portion basis. Out of the portions, the portion applied with the least integral dose may be assigned as the irradiation area. In another case, an arbitrary portion designated by the radiological technician may be assigned as the irradiation area. A value calculated as the integral dose received by the irradiation area is not necessarily the mean value, but may be a maximum value, a mode value, or a sum value of the integral values of the dose detection signals of the detection pixels 41b present within the irradiation area.

The emission stop signal from the comparator 72 of the AEC circuit 60 is outputted to the emission signal I/F 61 (see FIG. 5) through the control circuit 48. The emission signal I/F 61 transmits the emission stop signal to the emission signal I/F 26 of the source controller 11 connected wiredly or wirelessly. Note that, if the output of the detection pixel 41b during the pre-exposure is low obviously due to an implant embedded in the patient's body, the AEC circuit 60 may judge abnormality and output the emission stop signal to interrupt the X-ray emission.

In the main exposure, as described in later, the stop timer 25 of the source controller 11 measures the elapsed irradiation time, and stops the X-ray emission as soon as the main irradiation time set in the main exposure condition has elapsed. In the FPD 30, continuation of the charge accumulation operation after the stop of the X-ray emission increases noise caused by the dark charge added to the image data. Thus, to reduce the noise, the FPD 30 preferably detects the stop of X-ray emission and shifts to the readout operation as quickly as possible. Therefore, the AEC circuit 60 is used for detecting the stop of X-ray emission in the main exposure in this embodiment. In this case, the detection pixel selector 70 selects one or more detection pixels 41b present within a directly exposed area to which the X-rays are directly applied to the imaging area 40 without passing through the patient's body. The integrator 71 does not integrate the dose detection signal, and outputs the dose detection signal inputted from the detection pixel selector 70 as-is to the comparator 72. The comparator 72 compares the dose detection signal with a predetermined emission completion threshold value. When the dose detection signal is lowered to the emission completion threshold value, the X-ray emission is judged to be completed.

Instead of the AEC circuit 60 having the function of detecting the completion of the X-ray emission, the FPD 30 may receive an emission completion signal, which indicates the completion of the X-ray emission, from the source controller 11. As an alternate way, if noise added to the image data is within an allowable range, the main irradiation time may be set on the control circuit 48 prior to the start of the charge accumulation operation of the FPD 30, and the FPD 30 may be shifted to the readout operation after a lapse of the main irradiation time.

The control circuit 48 has a timer 62. The timer 62 measures time between the transmission of the emission permission signal from the emission signal I/F 61 and the transmission of the emission stop signal from the emission signal I/F 61 in the pre-exposure, in other words, actual irradiation time (pre-irradiation time) in the pre-exposure. The pre-irradiation time is transmitted to a cassette controller 88 (see FIG. 10) of the console 14 through the communication I/F 55. The integral value of the dose detection signal at the moment of transmitting the emission stop signal, in other words, an actual integral dose (preliminary integral dose) applied during the pre-exposure is also transmitted to the cassette controller 88. Instead of above, the source controller 11 may measure the pre-irradiation time, and transmits the measured pre-irradiation time to the cassette controller 88 through the communication I/F 22.

As shown in FIG. 8, the console 14 stores the exposure conditions set for each body portion. Each exposure condition includes the tube voltage (in unit of kV), the tube current (in unit of mA), the irradiation area used for selecting the detection pixels 41b, the emission stop threshold value for judging the stop of X-ray emission during the pre-exposure, a necessary dose required for single radiographic imaging including the pre-exposure and the main exposure, and the like. The necessary dose takes such a value that the obtained X-ray image has high image quality adequate for diagnosis. The information about the exposure conditions is stored in the storage device 14c. The exposure condition corresponding to the body portion designated by the input device 14a is read out from the storage device 14c, and is provided for the electronic cassette 13 through the communication I/F 55. The radiological technician manually sets the exposure condition of the source controller 11 with referring to the exposure condition of the console 14.

The irradiation area is an area to be most noticed in diagnosis that is specified in each body portion, and an area from which the dose detection signal is obtained stably. In a case where the imaged body portion is the chest, for example, areas “A” and “B” that are enclosed by broken lines in FIG. 6A, that is, areas of the lung fields are assigned as the irradiation areas. Each irradiation area is represented by X and Y coordinates. In the case of the rectangular irradiation area, as with this embodiment, the X and Y coordinates of two points connected by a diagonal line are stored. The X and Y coordinates correspond to the positions of the pixels 41 (including the detection pixels 41b) in the imaging area 40. An X axis extends in a direction parallel to the scan lines 44, and a Y axis extends in a direction parallel to the signal lines 45. The coordinates of the most upper left pixel are assigned as an origin point (0, 0).

If noise added to the dose detection signal results in a low S/N ratio, the reliability of the main irradiation time that is determined based on the low S/N ratio is reduced. To secure the reliability, the target dose in the pre-exposure is preferably set high. On the other hand, the target dose has to be as low as possible in order to reduce the radiation exposure to the patient. For this reason, the target dose i.e. the emission stop threshold value is set at a minimum value, as long as the main irradiation time is reliably determined without being affected by various types of noise added to the dose detection signal.

As shown in FIG. 9, the console 14 is composed of a computer having the input device 14a, the monitor 14b, the storage device 14c, a CPU 75, the memory 76, and a communication I/F 77. These components are connected to each other via a data bus 78.

The storage device 14c is a hard disk drive (HDD), for example. The storage device 14c stores a control program and an application program 79. Running the application program 79 makes the console 14 perform various functions related to the radiography, such as display processing of the examination order and the X-ray image, image processing of the X-ray image, and setup of the exposure condition.

The memory 76 is a work memory used when the CPU 75 executes. The CPU 75 loads the control program stored on the storage device 14c into the memory 76, and runs the program for centralized control of the computer. The communication I/F 77 functions as a network interface for performing wireless or wired transmission control from/to an external device such as the RIS, the HIS, the image server, the source controller 11, and the electronic cassette 13.

As shown in FIG. 10, by running the application program 79, the CPU 75 of the console 14 functions as a storing and retrieving processing unit 85, an input/output controller 86, and a main controller 87. The storing and retrieving processing unit 85 stores various types of data to the storage device 14c, and retrieves the data from the storage device 14c. The input/output controller 86 reads out drawing data from the storage device 14c in response to operation on the input device 14a, and outputs to the monitor 14b various operation screens of GUIs based on the read drawing data. The input/output controller 86 receives input of operation commands from the input device 14a through the operation screens. The main controller 87, which includes the cassette controller 88 for controlling the operation of the electronic cassette 13 and a main exposure condition determination unit 89 for determining the main exposure condition, performs centralized control of the console 14. The functions described above may be embodied by hardware instead of software.

The cassette controller 88 receives from the storing and retrieving processing unit 85 information of the exposure condition corresponding to the body portion designated by the input device 14a, and provides the information to the electronic cassette 13 through the communication I/F 77. The cassette controller 88 receives the pre-irradiation time and a pre-integral dose, which corresponds to the integral value of the dose detection signal, from the electronic cassette 13 through the communication I/F 77.

The main exposure condition determination unit 89 receives information of the necessary dose corresponding to the set exposure condition from the storing and retrieving processing unit 85. The main exposure condition determination unit 89 obtains from the cassette controller 88 the pre-irradiation time and the pre-integral dose.

The main exposure condition determination unit 89 determines main irradiation time, being one item of the main exposure condition, based on the necessary dose, the pre-irradiation time, and the pre-integral dose. To be more specific, the pre-integral dose is divided by the pre-irradiation time, to obtain an X-ray dose received per unit of time in the pre-exposure. Since the pre-integral dose has already been applied to the body portion in the pre-exposure, the pre-integral dose is subtracted from the necessary dose. Then, this subtraction result is divided by the X-ray dose received per unit of time to obtain the main irradiation time. The main exposure condition determination unit 89 transmits information of the determined main irradiation time to the source controller 11 through the communication I/F 77. At this time, the main irradiation time itself may be transmitted, or a division of the main irradiation time by the pre-irradiation time (a ratio of the main irradiation time to the pre-irradiation time) may be transmitted.

As another item of the main exposure condition, a tube current-time product (main tube current-time product) in the main exposure may be determined instead of the main irradiation time. In this case, as in the case of determining the main irradiation time, the pre-integral dose is divided by the tube current-time product in the pre-exposure to obtain an X-ray dose received per unit of a tube current-time product in the pre-exposure. Then, the pre-integral dose is subtracted from the necessary dose, and this subtraction result is divided by the X-ray dose received per unit of the tube current-time product, to obtain the main tube current-time product. Information of the main tube current-time product is transmitted to the source controller 11. In this case, the main tube current-time product itself or a ratio of the main tube current-time product to the tube current-time product of the pre-exposure may be transmitted.

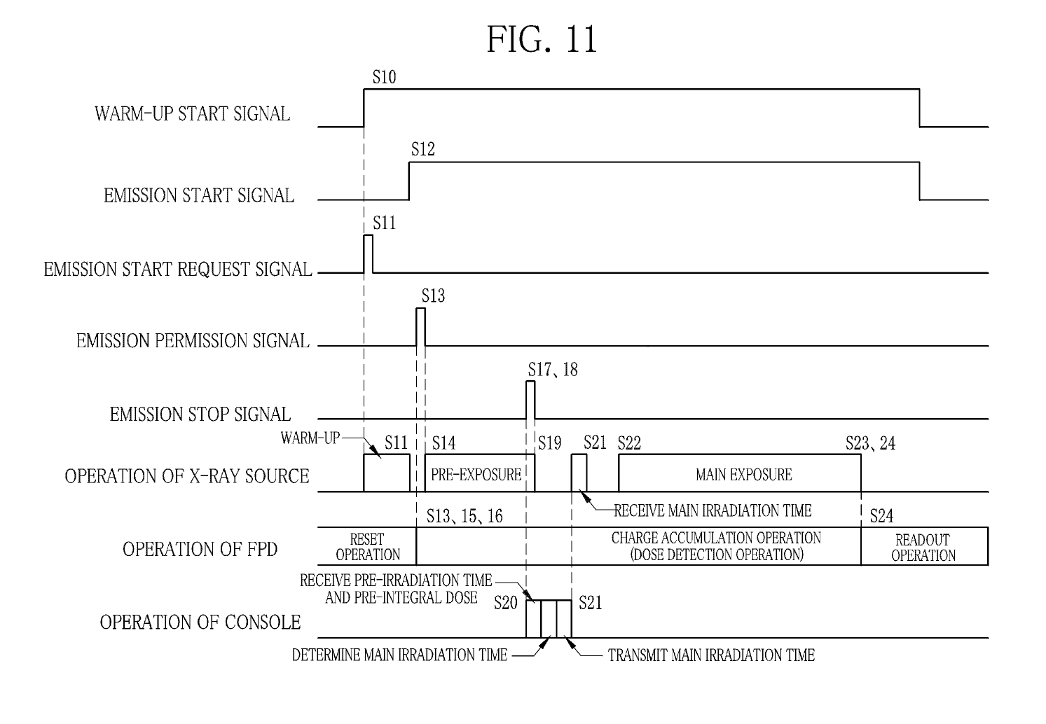

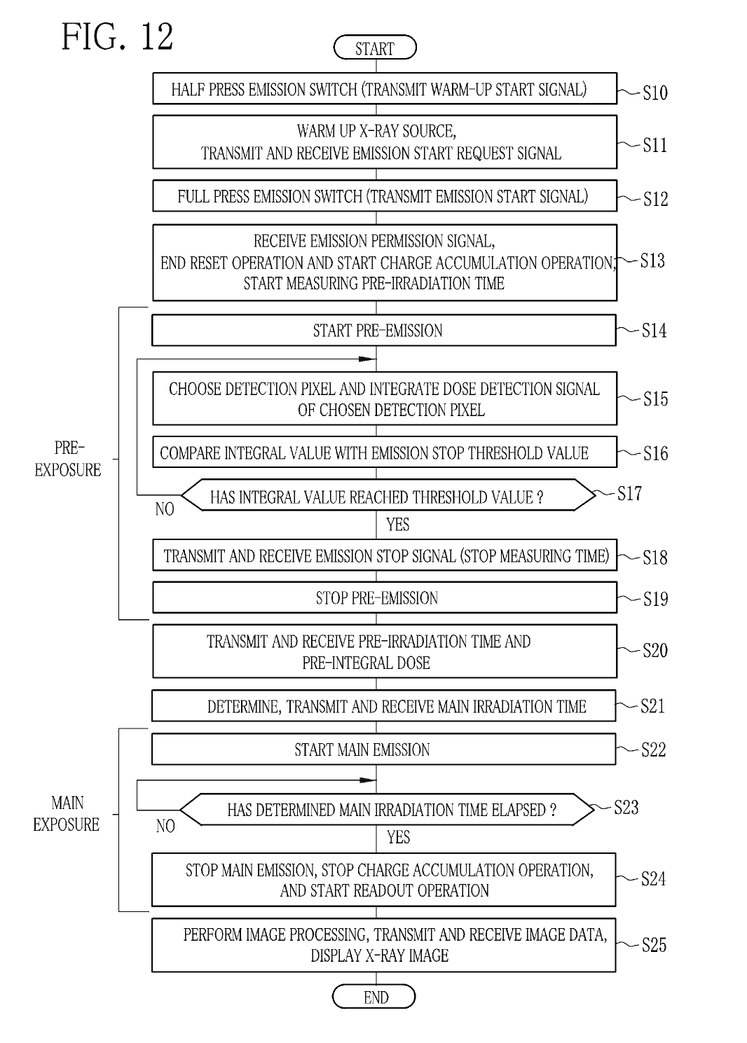

Next, the operation of the X-ray imaging system 2 will be described with referring to a timing chart of FIG. 11 and a flowchart of FIG. 12.

In performing the radiographic imaging with the X-ray imaging system 2, firstly, while the patient stands in a predetermined position in front of the imaging stand 15 or lies on the imaging table 16, the height and horizontal position of the electronic cassette 13 are adjusted with respect to the position of the body portion to be imaged. The height and horizontal position of the X-ray source 10 and the size of the irradiation field are adjusted in accordance with the position of the electronic cassette 13 and the size of the body portion. After that, the exposure condition is set on the source controller 11 and the console 14. The exposure condition set on the console 14 is provided to the electronic cassette 13.

After a preparation for the radiographic imaging is completed, the radiological technician half presses the emission switch 12. Thus, the warm-up start signal is transmitted to the controller 21 of the source controller 11 (S10 in FIGS. 11 and 12). The controller 21 starts warming up the X-ray source 10. The emission start request signal is transmitted from the emission signal I/F 26 of the source controller 11 to the emission signal I/F 61 of the electronic cassette 13 (S11). After the half press of the emission switch 12, when time required for the warming up has elapsed, the radiological technician fully presses the emission switch 12. Thereby, the emission start signal is issued to the controller 21 (S12).

In a standby mode, the control circuit 48 makes the FPD 30 repeat the reset operation. The emission signal I/F 61 waits for the emission start request signal from the emission signal I/F 26. When the emission signal I/F 61 receives the emission start request signal from the emission signal I/F 26 upon the half press of the emission switch 12, a state of the electronic cassette 13 is checked. After that, when the emission start signal is issued from the emission signal I/F 26 upon the full press of the emission switch 12 and the state check is completed, the emission permission signal is issued from the emission signal I/F 61 to the emission signal I/F 26. The FPD 30 finishes the reset operation and starts the charge accumulation operation and the dose detection operation, in other words, shifts from the standby mode to an exposure mode. The timer 62 starts measuring the pre-irradiation time (S13).

Upon receiving the emission permission signal by the emission signal I/F 26, the controller 21 makes the high voltage generator 20 start supplying electric power to the X-ray source 10 to perform the pre-exposure. Thereby, the pre-exposure is started (S14).

In the dose detection operation, the electric charge produced in the detection pixels 41b is read out a plurality of times at the predetermined sampling rate, and is converted into the dose detection signals by the A/D 52. The dose detection signals are transmitted to the AEC circuit 60. In the AEC circuit 60, the detection pixel selector 70 selects one or more dose detection signals of the detection pixels 41b present within the irradiation area out of the dose detection signals of all the detection pixels 41b inputted from the A/D 52, based on the information of the irradiation area provided by the console 14. The integrator 71 calculates the integral value (mean value) of the selected dose detection signal (S15). Then, the comparator 72 compares the integral value with the emission stop threshold value (S16).

When the integral value has reached the emission stop threshold value (YES in S17), the AEC circuit 60 judges that the pre-integral dose has come to the target dose, and issues the emission stop signal. The emission stop signal is transmitted from the emission signal I/F 61 to the emission signal I/F 26. At the same time, the timer 62 stops measuring the pre-irradiation time (S18). Upon receiving the emission stop signal, the source controller 11 stops the X-ray emission from the X-ray source 10 (S19). The FPD 30 continues the charge accumulation operation.

After the transmission of the emission stop signal, the electronic cassette 13 transmits the pre-irradiation time and the pre-integral dose to the cassette controller 88 of the console 14 (S20). The main exposure condition determination unit 89 determines the main irradiation time based on the necessary dose received from the storing and retrieving processing unit 85, the pre-irradiation time, and the pre-integral dose. The information of the determined main irradiation time is transmitted from the communication I/F 77 of the console 14 to the communication I/F 22 of the source controller 11 (S21).

Upon receiving the information of the determined main irradiation time, the controller 21 immediately sets the stop timer 25 at a value of the main irradiation time, and starts the electric power supply from the high voltage generator 20 to the X-ray source 10. Thereby, the main exposure is started (S22). Then, at the moment when actual irradiation time has come to the main irradiation time (YES in S23), the X-ray emission is stopped (S24).

The FPD 30 continues the charge accumulation operation throughout the pre-exposure and the main exposure. The AEC circuit 60 detects the completion of X-ray emission in the main exposure. If the AEC circuit 60 detects the completion of X-ray emission in the main exposure, the FPD 30 shifts from the charge accumulation operation to the readout operation (S24). Therefore, the image data representing the X-ray image of the single frame is outputted to the memory 54. After the readout operation, the FPD 30 returns the standby mode and repeats the reset operation.

The control circuit 48 applies various types of image processing to the X-ray image outputted in the readout operation to the memory 54. The X-ray image after the image processing is transmitted to the console 14 through the communication I/F 55 wiredly or wirelessly, and is displayed on the monitor 14b for use in diagnosis (S25). Thereby, the single radiographic imaging is completed.

As described above, no readout operation is performed in the pre-exposure, and the charge accumulation operation is continued from the start of the pre-exposure to the end of the main exposure. The main irradiation time is determined only based on the dose detection signals of the detection pixels 41b without output of any image, so it is possible to effectively use the X-ray dose applied during the pre-exposure. As a result, it is possible to reduce radiation exposure to the patient than before.

In a case where the pre-integral dose is reflected in the X-ray image for use in diagnosis, as described above, body movement between the pre-exposure and the main exposure possibly deteriorates the image quality of the X-ray image. According to the present invention, however, the main exposure is started immediately after the main irradiation time, which is determined by the main exposure condition determination unit 89, is transmitted from the console 14 to the source controller 11. Therefore, it is possible to reduce the adverse effect of the body movement to the X-ray image.

The pre-exposure continues until the pre-integral dose has reached the target dose, and the main irradiation time, being one item of the main exposure condition, is determined based on the necessary dose and the pre-integral dose and the pre-irradiation time detected during the pre-exposure. Therefore, the main exposure can be performed always with the appropriate exposure condition, irrespective of individual difference in physique of the patient, the density of the internal body tissue, and the like.

Since the AEC is carried out only in the pre-exposure, no problem caused by delay in the emission stop signal, that is, no problem of excessive irradiation time occurs in the main exposure. This prevents deterioration in the image quality of the X-ray image due to excessive radiation density and the unnecessary radiation exposure to the patient. Especially in the case of wirelessly transmitting and receiving the emission stop signal, the delay of the emission stop signal could become a serious problem. Depending on a radio state, the transmission and reception of the emission stop signal could fail and the stop of X-ray emission could be delayed significantly. According to the present invention, however, there is no such apprehension because the AEC is not performed in the main exposure. In this embodiment, the AEC is performed in the pre-exposure, so the delay of the emission stop signal could occur in the pre-exposure as a matter of course. However, since the target dose in the pre-exposure is set low, the delay of the emission stop signal occurring in the pre-exposure less affects the X-ray image and the patient, as compared with the case of performing the AEC in the main exposure.

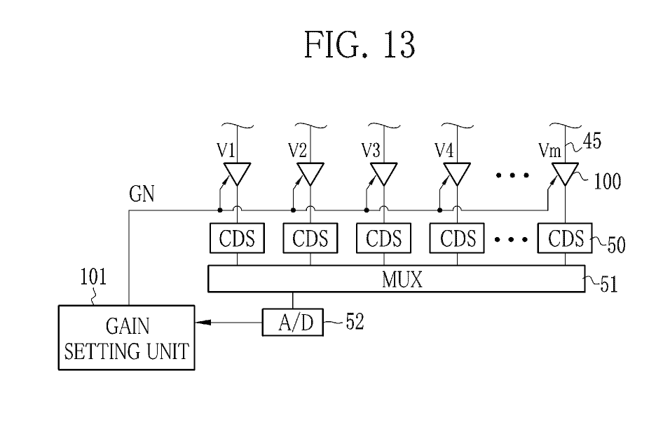

It is preferable that the gain of the amplifiers is set higher in the pre-exposure than in the readout operation. In this case, gain adjustable amplifiers 100 are used as shown in FIG. 13. This amplifier 100 amplifies an input voltage by feedback of the output of the operational amplifier to an input side. The gain of the amplifier 100 is adjustable by varying the ratio in a resistance value between input resistance (not shown) connected to an input terminal of the operational amplifier and feedback resistance (not shown) connected between the input and output terminals of the operational amplifier. To change the gain setting of the amplifiers 100, a gain setting unit 101 varies the resistance value of the input resistance and the resistance value of the feedback resistance in each amplifier 100 using a gain control signal GN.

Since the dose detection signal is sampled during the pre-exposure in a period of time much shorter than that of the charge accumulation operation during the main exposure, the dose detection signal is much smaller than the signal read out in the readout operation. However, the gain of the amplifier 100 is set higher in the pre-exposure than in the readout operation to amplify the dose detection signal to a larger value, so the S/N ratio of the dose detection signal inputted to the AEC circuit 60 is improved. Therefore, the AEC circuit 60 has the improved accuracy of exposure control.

The gain of the amplifier during the readout operation may be varied based on the dose detection signal. In this case, the structure and operation of the gain setting unit 101 are almost the same as those of the AEC circuit 60. The gain setting unit 101 starts the dose detection operation in synchronization with the start of the charge accumulation operation of the FPD. However, the gain setting unit 101 is different from the AEC circuit 60 in terms of continuing the dose detection operation even after the AEC circuit 60 stops the X-ray emission (pre-emission) in the pre-exposure, and finishing the dose detection operation at the moment when the AEC circuit 60 detects the end of the X-ray emission (main emission) in the main exposure using its emission completion detecting function.

The gain setting unit 101 integrates the dose detection signal of the detection pixel 41b present within the irradiation area from the start of the pre-emission to the end of the main emission. This integral value corresponds to the total integral dose applied during the pre-exposure and the main exposure, and is almost equal to a voltage signal V to be outputted from the normal pixel 41a present within the irradiation area. Thus, by making fine adjustment to the gain of the amplifier 100 during the readout operation based on this integral value, it is possible to obtain the X-ray image with high image quality invariably irrespective of variations in the total integral dose.

The gain setting unit 101 compares the above integral value with the necessary dose. When the integral value is much larger than the necessary dose, the gain setting unit 101 issues a gain control signal GN to lower the gain of the amplifier 100. On the other hand, when the X-ray dose received by the irradiation area is low and the integral value is the necessary dose or less, the gain control signal GN that commands the increase of the gain of the amplifier 100 is issued. At this time, the gain is determined such that maximum and minimum values of the voltage signal outputted from the normal pixel 41a present within the irradiation area fall within the range of the A/D converter. After the gain setting, the FPD 30 shifts to the readout operation.

When the exposure condition designates a low X-ray dose, the width between the maximum value and the minimum value of the voltage signal V is narrow with respect to the range of the A/D converter, and hence the obtained X-ray image becomes unclear with conspicuous noise. However, increasing the gain of the amplifier 100 makes it possible to obtain the X-ray image with high image quality without conspicuous noise. Therefore, the necessary dose is reduced, and as a result, the radiation exposure to the patient can be reduced.

The gain setting unit 101 carries out the dose detection operation from the start of the pre-emission until the end of the main emission, and switches the gain of the amplifier during the readout operation based on the integral value of the dose detection signal, which corresponds to the total integral dose over the pre-emission and the main emission. However, the dose detection operation may be carried out only in the pre-exposure. The gain of the amplifier may be adjusted based on the pre-integral dose and the main irradiation time or the main tube current-time product.

In this case, the pre-integral dose that is actually applied during the pre-exposure is the integral value being a measurement value, while the main integral dose that is actually applied during the main exposure is a predicted value calculated from the main irradiation time or the main tube current-time product. As a method for predict the main integral dose from the main irradiation time or the main tube current-time product, an expression or a data table for calculating an integral dose using irradiation time or a tube current-time product as a parameter is stored in advance in the storage device 14c of the console 14 and used. The sum of the calculated predicted value of the main integral dose and the measurement value of the pre-integral dose corresponds to the total integral dose received over the pre-exposure and the main exposure. Processing afterward is the same as that described above.

In a case where the gain of the amplifier is set higher in the pre-exposure than that in the readout operation, and the gain of the amplifier during the readout operation is changed based on the dose detection signal, the gain of the amplifier 100 is different between the dose detection operation in the pre-exposure and the dose detection operation in the main exposure. Thus, the integral value of the dose detection signal calculated by the gain setting unit 101 does not precisely represent the total integral dose received over the pre-exposure and the main exposure. In such a case, the dose detection signal obtained in the pre-exposure is preferably corrected so as to be equated with a value multiplied by the gain set in the main exposure. The integrating amplifier may be of a gain adjustable type, and performs gain adjustment.

In the above embodiment, the tube voltage takes the same value between the pre-exposure and the main exposure to apply the X-rays with unchanged radiation quality. However, in a case where pre-exposure takes much time due to low X-ray transmittance of the object, the tube voltage during the main exposure may be changed so as to improve the radiation quality of the X-rays (increase X-ray energy) in order to shorten the main irradiation time. The higher the radiation quality, the more X-rays pass through the object. Thus, the X-ray dose received by the FPD 30 is increased, and hence the irradiation time is shortened. Long irradiation time tends to cause deterioration in the image quality of the X-ray image due to the body movement, so the short irradiation time is preferable in consideration of the effect of the body movement.

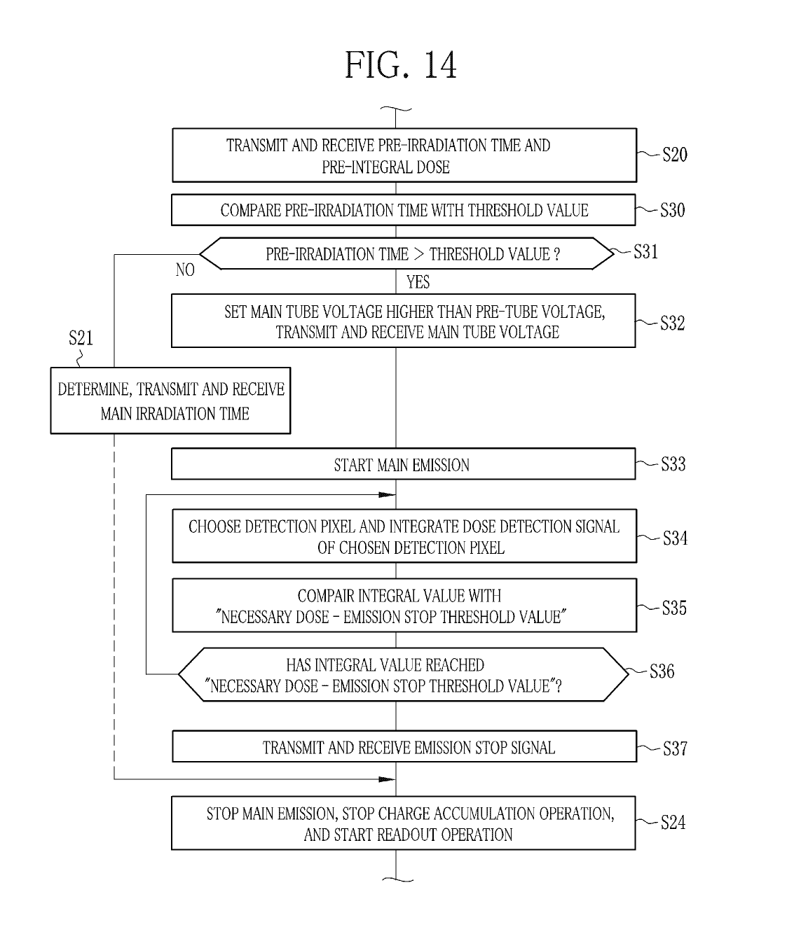

To be more specific, as shown in FIG. 14, the main exposure condition determination unit 89 compares the pre-irradiation time with a predetermined irradiation time threshold value (S30). When the pre-irradiation time is larger than the irradiation time threshold value (YES in S31), the tube voltage (main tube voltage) in the main exposure is changed to a higher value than that (pre-tube voltage) in the pre-exposure. Information of the main tube voltage is transmitted to the communication I/F 22 through the communication I/F 77 (S32). The irradiation time threshold value is determined such that when the pre-irradiation time is the irradiation time threshold value or more, the main irradiation time exceeds a limit of allowing the effect of the body movement. A plurality of irradiation time threshold values, which depend on the body portion to be imaged, the sex and age of the patient, and the like, are stored in advance in the storage device 14c. The irradiation time threshold value is changed in accordance with the body portion, because a chest usually moves widely due to a heart beat and breath, while a hand or fingers hardly moves. The irradiation time threshold value is changed in accordance with the age of the patient, because a child usually cannot stay still for long as compared with an adult.

Upon receiving the information of the main tube voltage changed by the main exposure condition determination unit 89 through the communication I/F 22, the controller 21 changes the setting of the high voltage generator 20. The controller 21 starts the electric power supply from the high voltage generator 20 to the X-ray source 10 to start the main emission (S33).

In the case of changing the tube voltage, in contrast to the above embodiment, the main irradiation time cannot be calculated based on the pre-irradiation time with the use of a calculation method that is on the precondition that the tube voltage takes the same value in both the pre-exposure and the main exposure. Thus, in the electronic cassette 13, the AEC circuit 60 performs the AEC (S34 to S37) in the main exposure, as with the pre-exposure (S15 to S18 of FIG. 12). In S35, however, the integral value of the dose detection signal is compared with an integral dose required in the main exposure, that is, the subtraction of the pre-integral dose from the necessary dose. As described above, setting the main tube voltage higher than the pre-tube voltage can shorten the main irradiation time, and prevent deterioration in the X-ray image caused by the body movement. In a case where the pre-irradiation time is the irradiation time threshold value or less (NO in S31), the steps of S21 and later are performed as with the above embodiment, so the description thereof is omitted.

The irradiation area may be designated based on an analysis result of the dose detection signals from all the detection pixels 41b, in cases where the AEC circuit detects the stop of the main emission using its emission completion detecting function, where the gain of the amplifier during the readout operation is changed based on the dose detection signal, and where the AEC is performed in the main exposure as with the case of FIG. 14.

In the case of performing the AEC in the pre-exposure, the case of changing the gain of the amplifier during the readout operation, or the case of performing the AEC in the main exposure, the detection pixel selector 70 provides the integrator 71 with a minimum value of the dose detection signals of all the detection pixels 41b. In other words, an area having the detection pixel 41b that outputs the minimum value of the dose detection signal is designated as the irradiation area. On the other hand, in a case where the AEC circuit detects the stop of the main emission using its emission completion detecting function, the detection pixel selector 70 provides the integrator 71 with a maximum value of the dose detection signals of all the detection pixels 41b. In other words, an area having the detection pixel 41b that outputs the maximum value of the dose detection signal is designated as the irradiation area.

The area having the detection pixel 41b that outputs the minimum value of the dose detection signal corresponds to a thickest body portion, and is probably an area to be most noticed in diagnosis. Therefore, in the AEC and the gain setting, this area is designated as the irradiation area. On the other hand, the area having the detection pixel 41b that outputs the maximum value of the dose detection signal is probably a so-called directly-exposed area upon which the X-rays are directly incident without passing through the object. Therefore, in the case of detecting the stop of the main emission with the emission completion detecting function, this area is designated as the irradiation area.

As described above, automatically switching the irradiation area based on the dose detection signal in accordance with an application makes it possible to improve accuracy in the AEC, the gain setting, and the emission completion detection. Also, narrowing the irradiation area to a certain range facilitates shortening processing time, as compared with the case of performing the AEC, the gain setting, and the emission completion detection based on the dose detection signals of all the detection pixels 41b without selecting the irradiation area. As a result, the X-ray imaging system 2 can smoothly stop the X-ray emission using the AEC, and the FPD 30 can smoothly shift from the charge accumulation operation to the readout operation in response to the emission completion detection.

In the above embodiment, the detection pixel has such structure that the photoelectric conversion element 42 is directly connected to the signal line 45 without through the TFT 43. However, the detection pixel may have the TFT 43 having a short between the source electrode and the drain electrode, or may have another TFT that is driven independently of the TFT 43.

In the above embodiment, the detection pixel is approximately the same size as the single normal pixel, and the detection pixels substitute for the several normal pixels in the matrix of pixels. However, the size of the detection pixel is arbitrarily changeable. The detection pixel may be smaller than the single normal pixel, or may have a size of a plurality of normal pixels. The detection pixel may be disposed between the normal pixels adjoining to each other. The detection pixel is not necessarily in a square shape, may be rectangular.

With taking advantage of the fact that electric current flowing through the bias line, which supplies the bias voltage to each pixel, is in proportional to the amount of the electric charge produced in the pixel, the electric current flowing through the bias line connected to the specific pixel may be monitored to detect the received X-ray dose. In this case, the pixel the electric current of which is monitored is designated as the detection pixel.

Furthermore, the detection pixel that is formed in a manufacturing process similar to that of the normal pixel is used as a dose detection sensor. However, another dose detection sensor that has different structure, material, manufacturing process, and the like from those of the normal pixel may be used.

However, it is difficult to form the dose detection sensors having structure different from that of the normal pixels in the imaging area, because the manufacturing process of the dose detection sensors is different from that of the normal pixels. In consideration of ease of manufacturing, the detection pixels that can be formed in the manufacturing process similar to that of the normal pixel, as described in the above embodiment, are preferably used as the dose detection sensors. The dose detection sensor is not necessarily formed in the imaging area of the FPD, and a dose detection sensor separate from the FPD may be used, as described in Japanese Patent Laid-Open Publication No. 2008-086358. However, the provision of the dose detection sensor separate from the FPD causes increase in size and manufacturing cost. Thus, as described in the above embodiment, the dose detection sensor is preferably the detection pixel formed in the imaging area of the FPD.

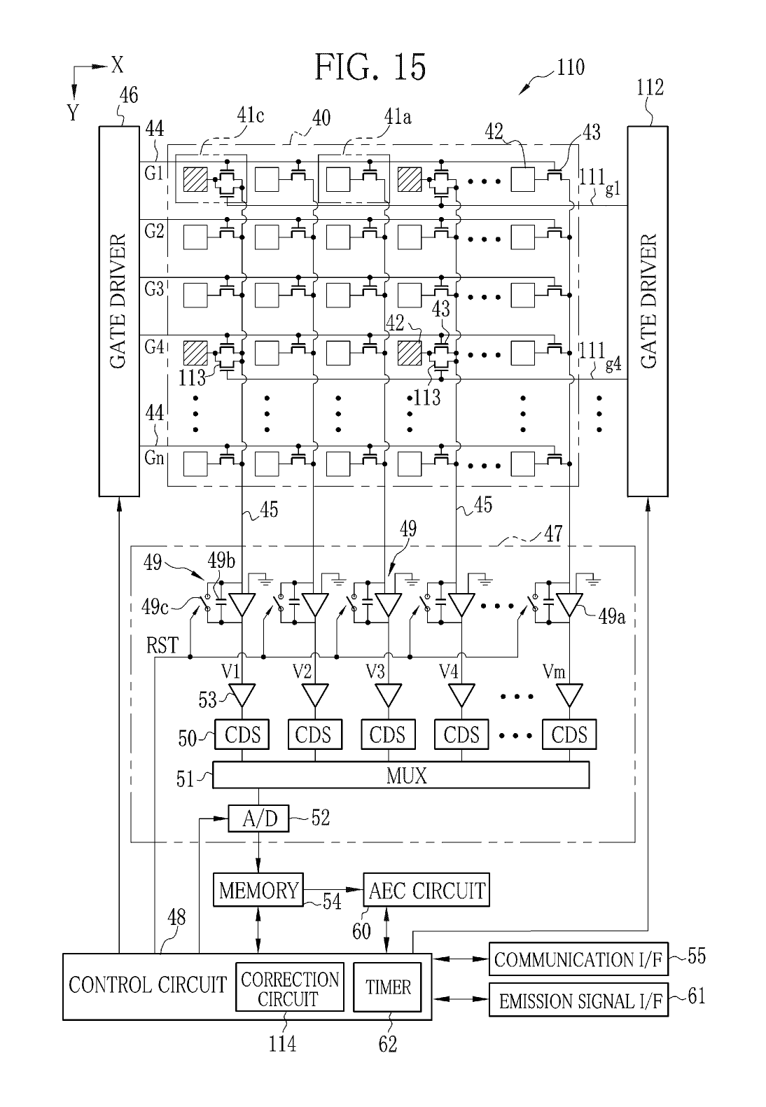

FIG. 15 shows another example of the detection pixels. The reference numerals same as those of the above embodiment refer to the components same as above, and the description thereof will be omitted.

In an FPD 110, a detection pixel 41c is provided with not only the TFT 43 that is driven by the scan line 44 and the gate driver 46, but also another TFT 113 that is driven by a scan line 111 and a gate driver 112. The single detection pixel 41c is provided in three-by-three i.e. nine pixels 41. Since the detection pixel 41c is provided with the TFT 113, the electric charge can be read out from the detection pixel 41c, even if the TFTs 43 are turned off and the normal pixels 41a in the same row as that of the detection pixel 41c are in the middle of the charge accumulation operation.

In the dose detection operation, the gate driver 112 transmits the emission permission signal to the source controller 11 under the control of the control circuit 48. In synchronization with a shift of the FPD 30 from the standby mode for repeating the reset operation to the exposure mode for starting the charge accumulation operation, the gate driver 112 sequentially issues gate pulses g1, g4, g7, . . . , and gk (k=1+3(n−1)) each for driving the TFTs 113 of the same row at a time at predetermined intervals, so the scan lines 111 are sequentially activated one by one. Thus, the TFTs 113 connected to the scan lines 111 are turned on sequentially on a row-by-row basis, and this operation is repeated at a predetermined sampling rate. As an alternative way, the TFTs 113 of the detection pixels 41c that are present within the irradiation area are selectively turned on. By turning on the TFT 113, the electric charge produced in the photoelectric conversion element 42 of the detection pixel 41c flows into the capacitor 49b of the integrating amplifier 49 through the signal line 45. Processing after this is the same as that of the above embodiment, so the description thereof will be omitted.

In the cases of the detection pixel 41b having the photoelectric conversion element 42 directly connected to the signal line 45 without through the TFT 43 and the detection pixel having the TFT 43 having a short circuit across the source electrode and the drain electrode, the detection pixel cannot accumulate the electric charge produced in the photoelectric conversion element 42, so the pixel value of the detection pixel is necessarily corrected by the defect correction circuit. However, in the case of the FPD 110 in which the electric charge accumulated in the detection pixels 41c can be read out independently of the readout of the normal pixels 41a, turning off both the TFTs 43 and 113 allows accumulating the electric charge in the detection pixel 41c. Thus, it is possible to obtain pixel values of the detection pixels 41c without totally recourse to the defect correction, which corrects a pixel value of a defect pixel using a pixel value of a normal pixel near the defect pixel by linear interpolation.

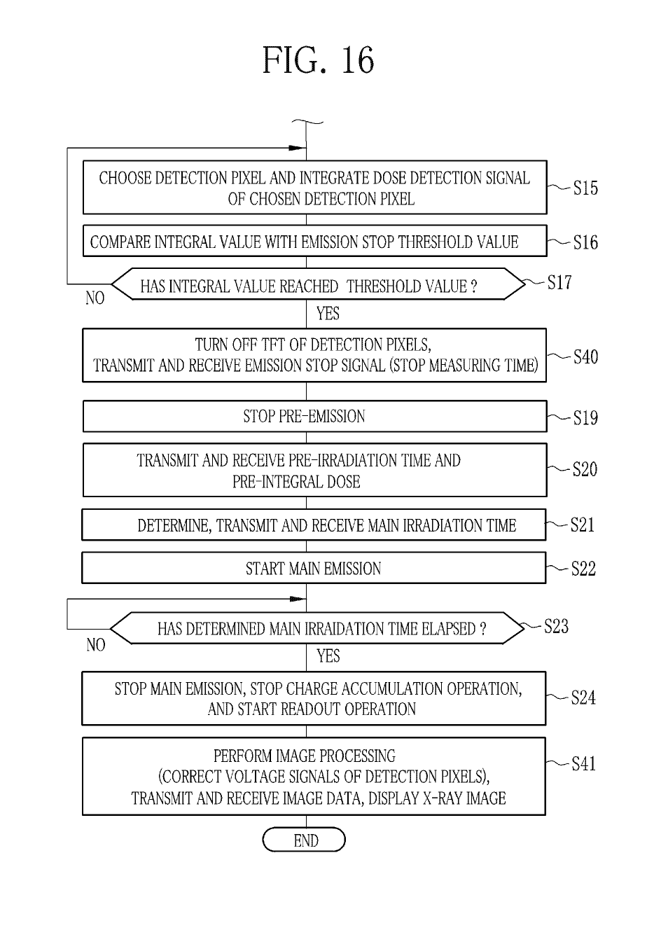

In the case of using the FPD 110, if it is judged that the integral dose detected by the detection pixel 41c has reached the target dose, as shown in S40 of FIG. 16, the TFTs of the detection pixels 41c are turned off so that the detection pixels 41c accumulate the electric charge produced in the main exposure following thereto. In the readout operation, the electric charge accumulated in the detection pixels 41c is read out together with the electric charge of the normal pixels 41a as voltage signals (S24).

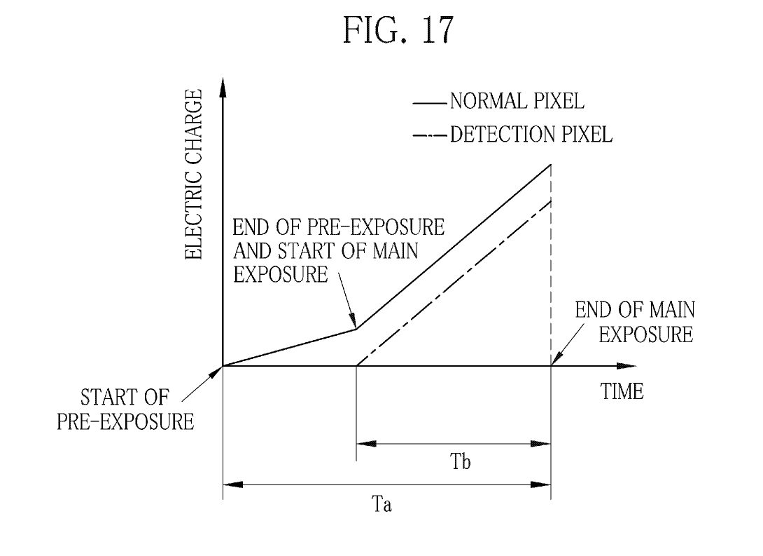

In this case, however, as shown by an alternate long and short dashed line in FIG. 17, only the electric charge produced in the main exposure is reflected in the voltage signal outputted from the detection pixel 41c in the readout operation. The electric charge produced in the pre-exposure is used in the AEC, and hence is not reflected in the voltage signal. For this reason, a value of the voltage signal of the detection pixel 41c is less than that of the normal pixel 41a by an amount of the electric charge produced in the pre-exposure. A correction circuit 114 (see FIG. 15) multiplies the voltage signals outputted from the detection pixels 41c by the ratio of the sum of the pre-irradiation time and the main irradiation time to the main irradiation time, in other words, the ratio Ta/Tb of a charge accumulation period Ta of the normal pixels 41a to a charge accumulation period Tb of the detection pixels 41c (S41 of FIG. 16). This correction is based on the voltage signals that are actually outputted from the detection pixels 41c, and hence is more reliable than the defect correction in which the pixel value of the defect pixel is created from nothing by the linear interpolation. The output of the detection pixels 41c is used for production of the X-ray image, and deterioration in the image quality due to the provision of the detection pixels 41c is minimized. In the case of selectively turning on the TFTs 113 of the detection pixels 41c present within the irradiation area, the detection pixels 41c present outside the irradiation area are shifted to the charge accumulation operation together with the normal pixels 41a in synchronization with the start of the pre-exposure. The console 14 may be provided with the correction circuit 114, and make a correction to the voltage signal from the detection pixels 41c.

In the above embodiment, when the integral value of the dose detection signal has reached the emission stop threshold value, it is judged that the integral dose has reached the target dose, and the emission stop signal is outputted. However, a predicted time at which the integral dose is to reach the target dose may be calculated instead. The emission stop signal may be transmitted to the source controller at the moment of reaching the predicted time, or information of the predicted time itself may be transmitted to the source controller. In this case, the source controller measures actual irradiation time, and stops the X-ray emission at the moment when the actual irradiation time has come to the predicted time. In the case of using the FPD 110 of FIG. 15, as soon as the predicted time has been calculated, the detection pixel 41c that has been outputting the dose detection signal is shifted to the charge accumulation operation by turning off its TFT 113. Thus, the electric charge produced during the pre-exposure is used for production of the X-ray image with minimum wastage.

In the above embodiment, the X-ray image that has been applied to various types of image processing in the control circuit 48 and is to be used in diagnosis is outputted to the console 14 and displayed on the monitor 14b after the main exposure. However, as described below, a preview image may be displayed before the display of the X-ray image.

In the above embodiment, the detection pixels 41b are uniformly distributed in the entire imaging area 40, and the dose detection signals from the detection pixels 41b are stored in the memory 54 with being associated with the coordinates of each detection pixel 41b. Thus, image data stored in the memory 54 cannot be used in diagnosis because of low resolution, but is usable for checking a state of the patient or the object. Therefore, by a preview of the image data based on the dose detection signals, the radiological technician can check the inappropriateness of the position of the patient or the object in a case where the patient's body moves during the pre-exposure.

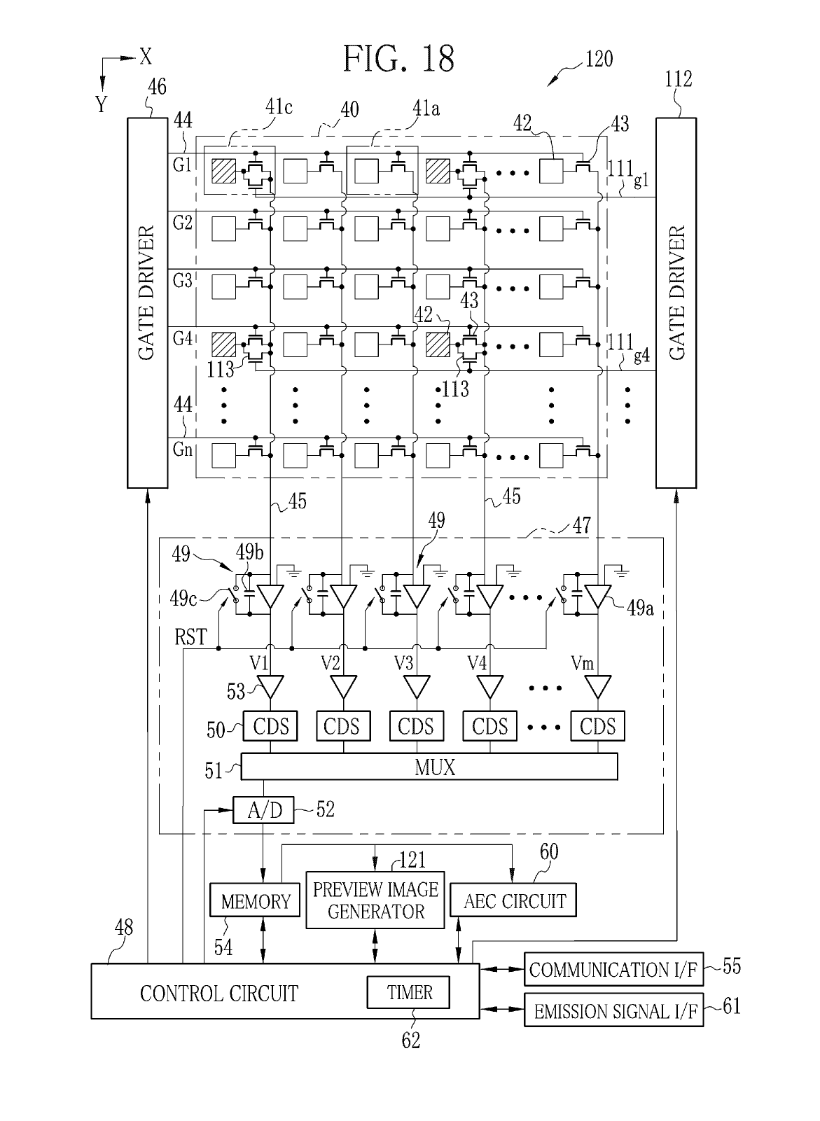

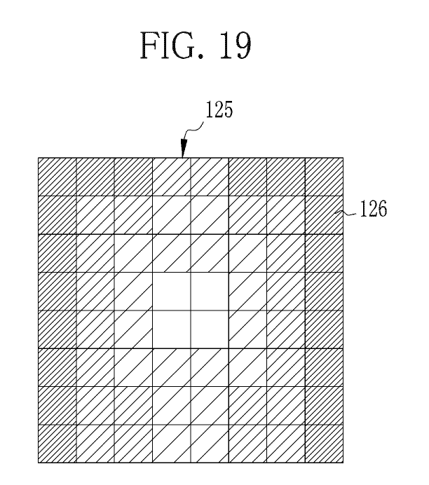

In FIG. 18, an FPD 120 has a preview image generator 121. The other fundamental structure of the FPD 120 is the same as that of the FPD 110 of FIG. 15. The preview image generator 121 produces a preview image 125, as shown in FIG. 19, from the dose detection signals that are outputted from the detection pixels 41c during the dose detection operation in the pre-exposure.

As shown in FIG. 19, the preview image 125 represents the amount of the X-ray dose received by the imaging area 40 on the basis of each of divided portions 126 into which the imaging area 40 is equally divided. Each divided portion 126 includes a plurality of normal pixels 41a and at least one detection pixel 41c. The preview image generator 121 calculates the integral value (mean value, maximum value, mode value, or sum value) of the dose detection signal or signals from one or more detection pixels 41b present within each divided portion 126. Furthermore, the preview image generator 121 integrates the integral value of the dose detection signal or signals of each divided portion 126 obtained for several times at the predetermined sampling rate. The preview image generator 121 produces the preview image 125 with regarding the divided portions 126 as pixels, and regarding the integral value of the dose detection signal or signals of each divided portion 126 as a pixel value.

The narrow-hatched divided portions 126 that correspond to the directly exposed area to which the X-rays are directly applied without through the object have the large integral values. The non-hatched divided portions 126 that correspond to an area to which the X-rays are applied through a relatively thick body portion, out of an object area on which the X-rays are incident through the object, have the small integral values. The wide-hatched divided portions 126 that correspond to the boundary between the directly exposed area and the object area and an area to which the X-rays are applied through a relatively thin body portion out of the object area have the middle integral values.

The preview image generator 121 transmits the produced preview image 125 to the console 14 through the communication I/F 55. The timing of the transmission of the preview image 125 to the console 14 is after the start of the main exposure, because if the preview image 125 is transmitted before the determination of the main exposure condition by the main exposure condition determination unit 89 and the start of the main exposure, the start of the main exposure is delayed. Also, the preview image 125 is transmitted before the readout operation of the FPD 120, because if the preview image 125 is transmitted during the readout operation of the FPD 120 after the main exposure, transmission noise tends to be added to the X-ray image, and may cause deterioration in the image quality of the X-ray image. For this reason, the preview image 125 is transmitted in the middle of the main exposure and during the charge accumulation operation of the FPD 120, for example.

The console 14 displays the preview image 125 on the monitor 14b until the X-ray image for use in diagnosis is transmitted from the FPD 120. The radiological technician checks the positioning of the object at the sight of the preview image 125. Compared with the case of transmitting and displaying an unprocessed X-ray image read out after the main exposure as the preview image, the preview image 125 is displayed more quickly, because the preview image 125 is produced from the dose detection signals outputted from the detection pixels 41c during the dose detection operation in the pre-exposure and is transmitted to the console 14 and displayed on the monitor 14b in the middle of the main exposure before the transmission of the X-ray image. The radiological technician checks the preview image 125 before the completion of the main exposure. If failure in the imaging is found out at the sight of the preview image 125 before the completion of the main exposure, the radiological technician releases the full press of the emission switch 12 to stop the X-ray emission.

The resolution of the preview image 125 is increased with increase in the number of the divided portions 126, but no resolution comparable to the resolution of the X-ray image for diagnosis is necessary as long as the position of the object is checkable on the preview image 125. The preview image 125 may be produced based on the mean value, the maximum value, the mode value, or the sum value of the dose detection signals obtained by a specific number-th sampling (for example, first sampling) or the integral value of the dose detection signals obtained by a first few samplings, instead of the integral value of the dose detection signals obtained by all the samplings performed between the start and the end of the pre-exposure. This allows production of the preview image 125 prior to the end of the pre-exposure, and speedup in the display of the preview image 125.

The preview image may be produced based on the dose detection signals outputted from the detection pixels 41c during the dose detection operation in the main exposure, instead of or in addition to in the pre-exposure.

As described below, the movement of the object may be detected from the preview images that are produced from the dose detection signals outputted from the detection pixels 41c during the dose detection operation in the pre-exposure and the main exposure.

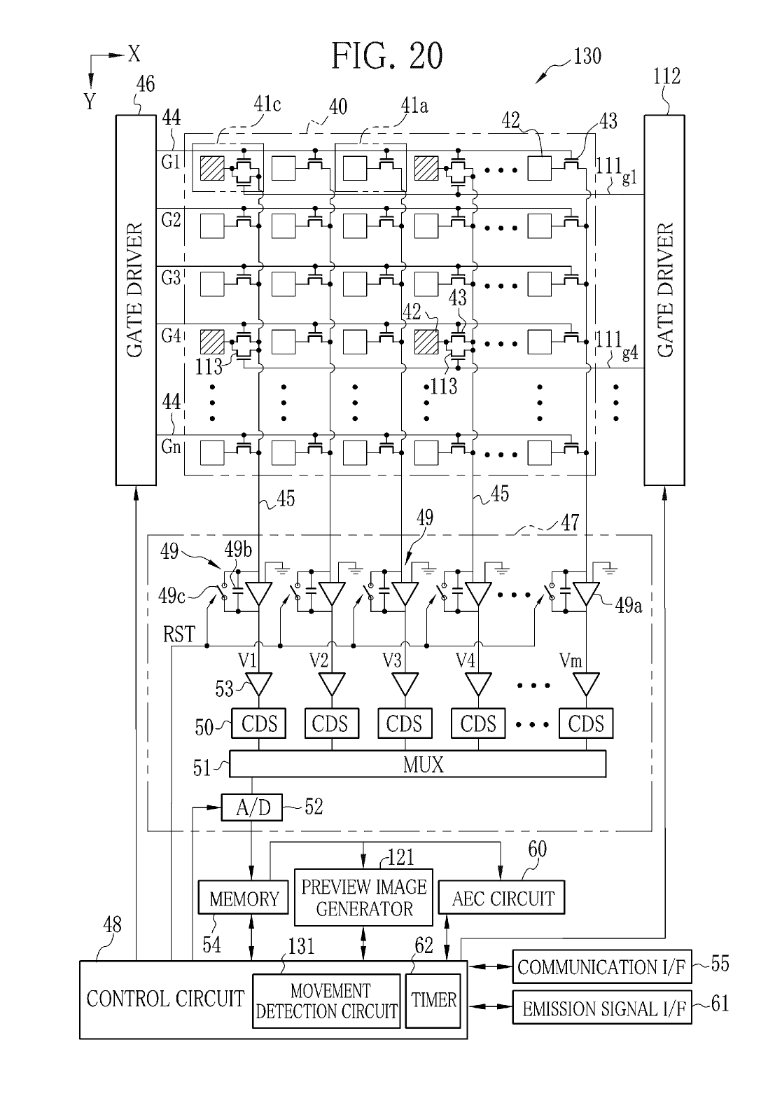

As shown in FIG. 20, in an FPD 130, the control circuit 48 has a movement detection circuit 131. The other structure of the FPD 130 is the same as that of the FPD 120 of FIG. 18. The preview image generator 121 produces the preview image based on the dose detection signals outputted from the detection pixels 41c during the dose detection operation in the pre-exposure, and the preview image base on the dose detection signals outputted from the detection pixels 41c during the dose detection operation in the main exposure. After the main exposure, the movement detection circuit 131 compares the preview images obtained in the pre-exposure and the main exposure, and quantitatively detects the movement of the object between the position of the pre-exposure and the position of the main exposure. In a case where the detected movement is larger than a predetermined movement threshold value, the movement detection circuit 131 transmits a movement detection signal to the console 14 through the communication I/F 55. When the detected movement is the movement threshold value or less, the movement detection circuit 131 does nothing.

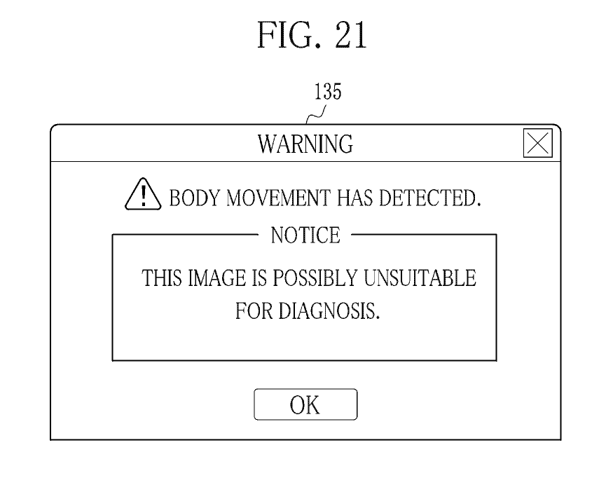

In response to the movement detection signal, as shown in FIG. 21, the console 14 displays on the monitor 14b a warning window 135 indicating a message that the X-ray image is possibly unsuitable for diagnosis due to the effect of the body movement. The warning window 135 notifies the radiological technician of the necessity of re-performing the imaging. Also, it is possible to prevent diagnosis from being performed based on the X-ray image having low image quality unsuitable for diagnosis.

The movement detection circuit 131 may detect the body movement of the object during the main exposure, instead of after the main exposure. In this case, the preview image generator 121 produces the preview image whenever one to several dose detection signals are sampled in the dose detection operation during the main exposure. The movement detection circuit 131 compares the preview image transmitted from the preview image generator 121 during the main exposure with the preview image produced during the pre-exposure, to detect the presence or absence of the body movement of the object, as with above. If the presence of the body movement is detected, the movement detection signal is transmitted to the console 14 as with above, and the transmission stop signal is transmitted to the source controller 11 through the emission signal I/F 61.

Upon receiving the movement detection signal, the console 14 displays the warning window 135 on the monitor 14b. When the emission signal I/F 26 receives the emission stop signal from the movement detection circuit 131, the controller 21 of the source controller 11 stops the electric power supply from the high voltage generator 20 to the X-ray source 10 to stop the X-ray emission. The presence or absence of the body movement of the object is detected in real time during the main exposure, and the X-ray emission is stopped during the main exposure if the presence of the body movement is detected. Thus, it is possible to prevent the unnecessary radiation exposure to the object.

In a case where the movement detection circuit 131 detects the body movement of the object, the X-ray image may not be outputted to the console 14 by disabling the readout operation of the FPD 130, abandoning data of the memory 54 after the readout operation, or the like. However, in a case where although the X-ray emission is stopped during the main exposure in response to the detection of the body movement by the movement detection circuit 131, the actual irradiation time is close to the main irradiation time, the X-ray image possibly has the image quality comparable to the image quality of the X-ray image obtained without detection of the body movement. Also, since the X-ray emission is stopped upon the detection of the body movement by the movement detection circuit 131, the body movement possibly has little effect on the X-ray image. For this reason, even if the movement detection circuit 131 detects the body movement of the object, the FPD 130 preferably performs the readout operation and outputs the X-ray image. The radiological technician judges whether or not the X-ray image has the image quality adequate for diagnosis.

The FPD 30 of FIG. 5 may adopt the production of the preview image and the detection of the body movement. In this case, however, the preview image is composed of strip-shaped divided portions that extend in the Y direction.

To indicate warning about the body movement of the object, for example, the electronic cassette 13 may sound a beep, turn on an LED lamp provided therein, or the like, instead of displays the warning window 135 on the monitor 14b as described above.

The console 14 and the electronic cassette 13 are separate in the above embodiment, but the console 14 may not be necessarily independent of the electronic cassette 13. The electronic cassette 13 may have the functions of the console 14. For example, the electronic cassette 13 may have the functions of the cassette controller 98 and the main exposure condition determination unit 99 of the above embodiment, and the electronic cassette 13 may determine the main irradiation time. Likewise, the source controller 11 and the console 14 may be integrated into one unit. Conversely, a specific imaging control device having the functions of the cassette controller 98 and the like may be provided between the electronic cassette and the console, and the console may be charged with only simple operations including the input of the exposure condition and the display of the X-ray image.

The present invention may be applied to a mounted type X-ray image detecting device, instead of the electronic cassette being a portable X-ray image detecting device. The present invention is applicable to a radiation imaging system using another type of radiation such as y-rays instead of the X-rays.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. A radiation imaging apparatus that performs main exposure for taking a radiographic image for use in diagnosis and pre-exposure prior to said main exposure for determining a main exposure condition set in said main exposure, said radiation imaging apparatus comprising:
an FPD having an imaging area having an arrangement of pixels each for accumulating electric charge in accordance with a radiation dose received by each of said pixels from a radiation generating apparatus through an object; and
a control unit for controlling operation of said FPD such that said FPD performs a charge accumulation operation continuously from start of said pre-exposure to end of said main exposure and performs a readout operation after the end of said main exposure, wherein
in said charge accumulation operation, said pixels accumulate said electric charge; and
in said readout operation, said accumulated electric charge is read out from said pixels and said radiographic image for use in the diagnosis is outputted.

2. The radiation imaging apparatus according to claim 1, further comprising:
a dose detection sensor for detecting said radiation dose received by said imaging area;
an AEC circuit for judging based on output of said dose detection sensor whether or not an integral dose being an integral value of said radiation dose has reached a target dose, and issuing an emission stop signal to stop radiation emission from said radiation generating apparatus when said integral dose is judged to have reached said target dose;
a timer for measuring pre-irradiation time from start of said radiation emission to issue of said emission stop signal in said pre-exposure;
a main exposure condition determination unit for determining said main exposure condition based on said pre-irradiation time and a predetermined necessary dose required for production of said radiographic image; and
a communication unit for transmitting said emission stop signal and said main exposure condition to said radiation generating apparatus.

3. The radiation imaging apparatus according to claim 2, wherein
said main exposure condition determination unit determines, as said main exposure condition, main irradiation time being radiation irradiation time in said main exposure or a main tube current-time product being a product of tube current in said main exposure and said main irradiation time; and
to determine said main irradiation time or said main tube current-time product, said main exposure condition determination unit divides a pre-integral dose being said integral value of said radiation dose in said pre-exposure by said pre-irradiation time or a tube current-time product in said pre-exposure to obtain an integral dose per unit of time or per unit of said tube current-time product, and then divides a subtraction result of said pre-integral dose from said necessary dose by said integral dose per unit of time or per unit of said tube current-time product.

4. The radiation imaging apparatus according to claim 2, further comprising:
an amplifier for amplifying both a voltage signal read out from said pixels in said readout operation and said output of said dose detection sensor in said pre-exposure with different gains; and
a gain setting unit for changing gain setting of said amplifier, said gain setting unit setting said gain to be used for amplifying said output of said dose detection sensor higher than said gain to be used for amplifying said voltage signal.

5. The radiation imaging apparatus according to claim 4, wherein
said gain setting unit has function of calculating at least one of said pre-integral dose and a main integral dose being said integral value of said radiation dose during said main exposure; and
said gain setting unit determines said gain used in said readout operation based on said pre-integral dose, said main integral dose, or a comparison result between said necessary dose and a sum of said pre-integral dose and said main integral dose.

6. The radiation imaging apparatus according to claim 2, wherein said main exposure condition determination unit determines tube voltage used in said main exposure based on a comparison result between said pre-irradiation time and a predetermined irradiation time threshold value.

7. The radiation imaging apparatus according to claim 6, wherein in a case where said main exposure condition determination unit changes said tube voltage used in said main exposure from said tube voltage used in said pre-exposure, said dose detection sensor and said AEC circuit perform exposure control in said main exposure.

8. The radiation imaging apparatus according to claim 2, further comprising:
a memory for storing which of a plurality of said dose detection sensors distributed in said imaging area to use on a basis of a body portion to be imaged;
an input device for designating said body portion; and
a detection sensor selector for selecting one or more of said dose detection sensors in accordance with said body portion designated by said input device.

9. The radiation imaging apparatus according to claim 8, wherein said detection sensor selector selects, out of said plurality of dose detection sensors, said dose detection sensor present in at least one of an area to be most noticed in diagnosis and a directly exposed area to which radiation is directly applied.

10. The radiation imaging apparatus according to claim 8, wherein said pixels include:
a normal pixel for producing and accumulating signal charge in accordance with said radiation dose and outputting said signal charge to a signal line through a switching element; and
a detection pixel functioning as said dose detection sensor, said detection pixel being directly connected to said signal line without through said switching element or being provided with another switching element driven independently of said switching element of said normal pixel.

11. The radiation imaging apparatus according to claim 10, wherein
in said pre-exposure, said detection pixel performs a dose detection operation for detecting said radiation dose; and
in said main exposure, said detection pixel performs said charge accumulation operation together with said normal pixel, and accumulated said signal charge is read out from both of said normal pixel and said detection pixel after completion of said charge accumulation operation.

12. The radiation imaging apparatus according to claim 11, further comprising:
a correction circuit for correcting an output value of said detection pixel in accordance with a ratio Ta/Tb between a charge accumulation period Ta of said normal pixel starting from said pre-exposure and a charge accumulation period Tb of said detection pixel starting from said main exposure, wherein
said control unit produces said radiographic image based on said corrected output value of said detection pixel and an output value of said normal pixel.

13. The radiation imaging apparatus according to claim 2, further comprising:
a preview image generator for producing a preview image based on output of said dose detection sensor in said pre-exposure; and
a console for receiving said preview image from said preview image generator and displaying said preview image, said preview image generator transmitting said preview image to said console before said FPD performs said readout operation.

14. The radiation imaging apparatus according to claim 13, wherein said preview image generator transmits said preview image to said console, while said FPD is performing said charge accumulation operation in said main exposure.

15. The radiation imaging apparatus according to claim 14, further comprising a movement detection circuit, wherein
said preview image generator produces a pre-preview image based on said output of said dose detection sensor in said pre-exposure, and produces a main preview image based on said output of said dose detection sensor in said main exposure;
said movement detection circuit compares said pre-preview image with said main preview image to detect presence or absence of body movement of said object between said pre-exposure and said main exposure; and
when said movement detection circuit detects the presence of the body movement, said movement detection circuit performs at least one of a step of outputting said emission stop signal to stop the radiation emission from said radiation generating apparatus and a step of warning the presence of the body movement.

16. The radiation imaging apparatus according to claim 2, wherein after said communication unit transmits said main exposure condition, said main exposure is started immediately.

17. The radiation imaging apparatus according to claim 2, wherein said communication unit adopts a wireless method.

18. The radiation imaging apparatus according to claim 1 being an electronic cassette having said FPD contained in a portable housing.

19. An operation method of a radiation imaging apparatus that performs main exposure for taking a radiographic image for use in diagnosis and pre-exposure prior to said main exposure for determining a main exposure condition set in said main exposure, said radiation imaging apparatus including an FPD having an imaging area having an arrangement of pixels and a control unit for controlling operation of said FPD, said operation method comprising the steps of:
accumulating electric charge in said pixels continuously from start of said pre-exposure to end of said main exposure;
reading out accumulated said electric charge from said pixels after the end of said main exposure: and
producing a radiographic image for use in diagnosis from said accumulated electric charge.

20. The operation method according to claim 19, said FPD including a dose detection sensor for detecting a radiation dose received by said imaging area, said operation method further comprising the steps of:
emitting radiation from a radiation generating apparatus to an object in said pre-exposure;
detecting, by said dose detection sensor, said radiation dose received by said imaging area through said object;
judging whether or not an integral dose being an integral value of said radiation dose has reached a target dose based on output of said dose detection sensor;
when said integral dose is judged to have reached said target dose, issuing an emission stop signal to stop the emission of said radiation from said radiation generating apparatus;
measuring pre-irradiation time from start of said pre-exposure to the issue of said emission stop signal;
determining said main exposure condition based on said pre-irradiation time and a predetermined necessary dose required for production of said radiographic image; and
transmitting said main exposure condition to said radiation generating apparatus.
